(19) United States
(12) Patent Application Publication
(10) Pub. No.: US 2003/0133912 A1
(43) Pub. Date: Jul. 17, 2003

(54) Receptor-targeted adenoviral vectors

(76) Inventors: Beverly L. Davidson, North Liberty, IA (US); Haibin Xia, Iowa City, IA (US); Lane K. Law, Iowa City, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/291,250

(22) Filed: Nov. 7, 2002

Related U.S. Application Data
(60) Provisional application No. 60/339,282, filed on Dec. 11, 2001.

(51) Int. Cl. A61K 48/00; C12N 7/00; C12N 15/861; C12N 5/08

(52) U.S. Cl. 424/93.2; 435/235.1; 435/366; 435/456; 435/320.1

(57) ABSTRACT

The invention provides adenoviral vectors possessing fibers that do not bind CAR, a receptor-targeting ligand sequence, the non-receptor targeting sequence derived from HIV-TAT, a sequence encoding the HI loop of the non-CAR binding adenovirus fiber, which permits specific receptor-targeted transduction with the recombinant adenovirus vectors. The invention also provides related chimeric proteins, adenovirus particles and mammalian cells.

### RELATED APPLICATIONS

This present application claims priority to U.S. Provisional Patent Application Serial No. 60/339,282, filed Dec. 11, 2001, which is incorporated herein by reference.

### GOVERNMENT FUNDING

Portions of the present invention were made with support of the United States Government via a grant from the National Institutes of Health under grants numbered HD33531 and DK54759. The U.S. Government therefore may have certain rights in the invention.

### BACKGROUND OF THE INVENTION

Adenoviruses (Ad) are double-stranded linear DNA viruses with a 36 kb genome. Adenoviruses have fiber proteins or “spikes” that protrude from each of the twelve vertices of the icosahedral capsid of the virus. The fiber protein consists of two domains: a rod-like shaft portion and a globular head portion that contains the putative receptor binding region. The fiber spike is a homotrimer. Adenoviruses interact with eukaryotic cells through specific receptor recognition of domains in the knob portion of the fiber protein, and thereby deliver its viral sequences to the target eukaryotic cells. Human adenoviruses may bind to and infect a broad range of cultured cell lines and primary tissues from different species.

Adenoviruses may be employed as delivery vehicles for introducing desired sequences into eukaryotic cells, such as sequences encoding a therapeutic protein or enzyme. Recombinant adenovirus vectors are used in a number of gene therapy applications, as high levels of gene transfer are possible both in vitro and in vivo. Unlike some other gene delivery systems, recombinant adenovirus vectors are capable of in situ gene delivery to differentiated target cells of a variety of organ types. This allows the use of recombinant adenoviral vectors to treat inherited genetic diseases, such as cystic fibrosis, where the delivered vector is targeted to a specific organ. This specificity also allows for the development if in situ tumor transduction to provide a variety of anti-cancer gene therapies for loco-regional diseases.

Adenoviral vectors accomplish in vivo gene delivery to a variety of organs after intravenous injection or direct inoculation into tissues. In these instances, gene transfer frequencies have been sufficiently high to correct inherited metabolic abnormalities in various murine models. Thus, adenoviral vectors fulfill two requirements of an intravenously administered vector for gene therapy: systemic stability and the ability to accomplish long-term gene expression following high efficiency transduction.

One disadvantage of the most commonly used adenovirus-based vectors, those derived from adenovirus type 5, however, is that the widespread distribution of the adenovirus cellular receptor precludes the targeting of specific cell types. This lack of tropism of adenoviral vectors would result in a decrease in the efficiency of transduction, as the number of virus particles available for delivery to the target cells would be decreased by sequestration by nontarget cells. Furthermore, this would allow ectopic expression of the delivered gene, with unknown and possibly deleterious consequences. Therefore, a means must be developed to redirect the tropism of the adenovirus vector specifically to target cells and permit gene delivery only to affected organs.

Adenovirus tropism is the result of specific binding of the virus to the target cell by means of a cellular receptor. The C-terminal portion of adenovirus fiber or “knob” is responsible for the specificity of receptor recognition. The 46-kDa human coxsackie-adenovirus receptor (CAR) interacts with the fiber knob of several adenoviral serotypes (2, 4, 5, 9, 12, 15, 17, 19, 31, 41), indicating that CAR can function as their cellular receptor. The mouse homologue of CAR (mCAR) has also been isolated and shown to mediate infection of the human adenovirus Ad2.

CAR is a type I transmembrane protein containing a cytoplasmic COOH-terminus and an N-terminal extracellular domain. The exoplasmic region contains two immunoglobulin-related domains (IgV and IgC2), stabilized by intrachain disulfide bonds and two potential N-linked glycosylation sites. Ad5 fiber interactions with CAR occur within the N-terminal IgV domain. Prospective amino acid residues within fiber required for binding to CAR have been identified. Using competitive assays with recombinant fiber it was found that two amino acids in the AB loop (Ser₄₀₈ and Pro₄₀₉), one in the DE loop (Tyr₄₇₇) and one in the FG loop (Tyr₄₉,) were essential. More recent surface plasmon resonance (SPR) binding studies with recombinant wild-type and mutant fibers and immobilized CAR unveiled the importance of Ala₄₀₆ and Arg₄₁₂ in the AB loop and Arg₄₈₅ in β strand F.

CAR is a putative cell adhesion molecule implicated to have a role in the developing brain. There is a high degree of sequence identity within the tail region of human and murine CAR. Interestingly, the tail and transmembrane domains are unnecessary for adenoviral binding and entry. In some instances, deletion or alteration of these sequences increased cell-surface expression of CAR leading to increased adenoviral-mediated gene transfer.

Several groups have reported genetic modifications to the knob domain of adenovirus fiber protein and incorporation of such chimeric fibers into virions. For instance, Stevenson et al., J. of Virology 69:2850-2857, (1995) reported successful generation of Ad5 virions containing fibers consisting of the tail and shaft domains of Ad5 fiber and the knob domain of Ad3, respectively. A US Patent issued to McClelland et al, U.S. Pat. No. 5,543,328 (“the '328 patent”), describes adenoviruses having modified fiber proteins. In this patent, at least a portion of the adenovirus fiber protein was removed, and then replaced with a ligand that is specific for a receptor located on a desired cell type. Specifically, they described the removal of a portion of the head region. This patent, however, does not discuss modification of non-CAR binding fiber proteins, and therefore permits specific adenovirus receptor-targeted transduction.

U.S. Pat. No. 5,547,932 describes conjugating an “internalizing factor” containing an endosomolytic agent to assist with the introduction of nucleic acid complexes into eukaryotic cells. U.S. Pat. No. 6,210,946 describes modifying an adenoviral fiber protein by replacing a portion of the native fiber protein with fibritin of bacteriophage T4. U.S. Pat. No. 5,871,727 uses a neutralizing anti-fiber antibody linked to the adenoviral fiber cell-binding protein to modify the tropism of recombinant adenoviral vectors.

There is a continuing need for improved adenovirus vectors that do not bind to CAR, and therefore permit specific adenovirus receptor-targeted transduction.

### SUMMARY OF THE INVENTION

The present invention provides an adenoviral vector comprising an adenoviral backbone encoding an adenoviral fiber that does not bind CAR and an adenoviral fiber protein HI-loop operably linked to a receptor-targeting ligand to form a ligand/HI-loop chimeric protein, wherein the chimeric protein binds to a corresponding targeted receptor but does not bind CAR. As used herein, “corresponding targeted receptor” is defined as a receptor that binds to the receptor-targeting ligand. For example, transferrin could be the receptor-targeting ligand, and the transferrin receptor would then be the corresponding targeted receptor. The present invention also provides an adenovirus particle or mammalian cell containing the above-described fiber.

The present invention also provides a chimeric protein comprising an amino acid sequence encoding an adenovirus fiber protein HI-loop operably linked to an amino acid sequence encoding a receptor-targeting ligand, wherein the chimeric protein binds to a corresponding targeted receptor but does not bind CAR.

The present invention provides a method of transducing cells lacking CAR comprising contacting the cells with the above-described vector or the above-described adenovirus particle.

The present invention further provides methods of treating a genetic disease or cancer in a mammal comprising administering the vector, the chimeric protein, the adenovirus particle, or the mammalian cell described above.

In general, the invention relates to polypeptides that can be used as a therapeutic agent, and polynucleotides, expression vectors, virus particles and genetically engineered cells, and the use of them for expressing a therapeutic agent. In particular, the invention may be used as a method for gene therapy that is capable of both localized and systemic delivery of a therapeutically effective dose of the therapeutic agent.

According to one aspect of the invention, a cell expression system for expressing a therapeutic agent in a mammalian recipient is provided. The expression system (also referred to herein as a “genetically modified cell”) comprises a cell and an expression vector for expressing the therapeutic agent. The expression vector further includes a promoter for controlling transcription of the heterologous gene. The promoter may be an inducible promoter. The expression system is suitable for administration to the mammalian recipient. The expression system may comprise a plurality of non-immortalized genetically modified cells, each cell containing at least one recombinant gene encoding at least one therapeutic agent.

The cell expression system can be formed ex vivo or in vivo. To form the expression system ex vivo, one or more isolated cells are transduced with a virus or transfected with a nucleic acid or plasmid in vitro. The transduced or transfected cells are thereafter expanded in culture and thereafter administered to the mammalian recipient for delivery of the therapeutic agent in situ. The genetically modified cell may be an autologous cell, i.e., the cell is isolated from the mammalian recipient. The genetically modified cell(s) are administered to the recipient by, for example, implanting the cell(s) or a graft (or capsule) including a plurality of the cells into a cell-compatible site of the recipient.

According to yet another aspect of the invention, a method for treating a mammalian recipient in vivo is provided. The method includes introducing an expression vector for expressing a heterologous gene product into a cell of the patient in situ. To form the expression system in vivo, an expression vector for expressing the therapeutic agent is introduced in vivo into target location of the mammalian recipient by, for example, intratumoral injection.

The expression vector for expressing the heterologous gene may include an inducible promoter for controlling transcription of the heterologous gene product. Accordingly, delivery of the therapeutic agent in situ is controlled by exposing the cell in situ to conditions that induce transcription of the heterologous gene.

Ad30 has been described in detail in U.S. Ser. No. 09/758,008 filed on Jan. 9, 2001, and is incorporated in its entirety by reference herein.

These and other aspects of the invention as well as various advantages and utilities will be more apparent with reference to the detailed description of the invention and to the accompanying drawings.

### BRIEF DESCRIPTION OF THE FIGURES

FIG. 1. Comparison of the amino acid sequence of adenoviral fibers from the subgroup D Ads (serotypes 30 (SEQ ID NO:1), 9 (SEQ ID NO:3), and 17 (SEQ ID NO:4) with those from subgroup C (serotype 5 (SEQ ID NO:2)) and subgroup B (serotypes 35 (SEQ ID NO:5) and 3 (SEQ ID NO:6)). The amino acid positions are based on Ad5. Blocked letters indicate amino acids found to be important for CAR binding (residues 406, 408, 409, 412, 417, 477, 481, 485).

FIG. 2. Schematic representation of homologous recombination in BJ5183 cells to generate pAd5GFPf30.

FIG. 3. Analysis of Ad5GFP and Ad5GFPf30 fibers by Western blot analysis. Purified Ad5GFP and Ad5GFPf30 were subjected to SDS PAGE, and viral proteins were transferred to nitrocellulose membranes. Membranes were incubated with a primary antibody to the N-terminus of Ad5 fiber followed by a peroxidase-conjugated goat anti-mouse secondary antibody. Membranes were developed with enhanced-chemiluminescence reagent. The gel is representative of at least three independent experiments from different viral isolates.

FIG. 4. The effects of CAR expression on Ad5GFP and Ad5GFPf30-mediated transduction. CHO cells were transfected with Ad5lacZ- or Ad5hCAR-CaPᵢ coprecipitants as described in the Examples below. Twenty-four hours later, transfected and non-transfected cells were infected with Ad5GFP or Ad5GFPf30 (500 particles/cell), 30 min at 37° C., followed by 24 hr. incubation. Cells were subsequently harvested and GFP expression analyzed by FACS analysis. Data represent mean±standard deviation from two independent experiments performed in triplicate.

FIG. 5. Effect of CAR on Ad5GFP and Ad5GFPf30 virus binding to CHO cells. CHO cells were transfected with AdhCAR as described in the legend to FIG. 4, or mock transfected. Cells were then incubated with ³H-labeled Ad5GFP, Ad5GFPf30 or wild-type Ad30 for 1 h on ice. Cell-associated radioactivity was determined and the number of viral particles bound per cell was calculated. The data are means±standard deviations and are representative of five independent experiments. Two independent viral isolations were used in these studies.

FIG. 6. Phage expressing the hTfR-targeting motifs B1 or B2 bind immobilized hTfR. Amplified, purified phage containing B1, B2, or random peptide (R) were tested for their ability to bind hTfR on 96-well microtiter plates. Bound phage were detected using anti-fd antibodies as described in Materials and Methods. The data represent the means±the SEM and are representative of three independent experiments.

FIG. 7. Peptide motif B2 binds immobilized hTfR. B2 peptide was synthesized with an amino-terminal biotin moiety and tested for binding specificity to hTfR using an ELISA-based assay. (A) Soluble hTfR (3.75 μg) was coated onto ELISA plates, incubated with biotin-labeled B2 at various concentrations, and detected using extravidin. (B) Soluble hTfR at various concentrations was coated onto plates, followed by incubation with a constant concentration of B2-biotin (25 μg). Plates were developed using extravidin-horseradish peroxidase, followed by detection of the optical density at 490 nm as described in the text. The data represent the means±the SEM from three independent experiments.

FIG. 8. Construction of recombinant adenoviruses with hTfR-targeting motifs in the HI loop. A series of HI loop-modified shuttle plasmids were generated (pBS/BxHI). These shuttles were cut with BamHI and NotI, and the 4.6-kb fragment was cotransformed with SwaI-cut pTG3602RSVGFP/SwaI into E. coli BJ5183. Appropriate recombination resulted in full-length Ad5 genomes with a GFP expression cassette in the E1 region and B1, B2, etc., motifs in the HI loop. The recombinant plasmids were restricted with PacI and transfected into HEK 293 cells, and virus was harvested and propagated upon finding evidence of cytopathic effect.

FIG. 9. Transduction of hTfR CHO cells by adenoviruses genetically modified to express hTfR-targeting epitopes. hTfR⁺ CHO cells (1×10⁵) were infected by Ad5GFPB1HI, Ad5GFPB2HI, etc., or Ad5GFP (4×10⁸ particles) for 1 h at 37° C., and GFP-positive cells were quantitated by FACS 48 h later For blocking studies, cells were incubated with soluble hTfR (sTfR) or transferrin for 30 min at 4° C. prior to the addition of virus. The various viruses are indicated by the TfR-targeting epitope (B1, B2, etc.). “C” corresponds to Ad5GFP. The data are the means±the SEM from three independent experiments.

FIG. 10. hTfR expression and transduction of T24 cells by hTfR-targeting epitope modified adenoviruses. T24 cells (1×10⁵) were transduced with Ad5GFPB5HI, Ad5GFPB6HI, etc., or Ad5GFP (4×10⁸ particles), and GFP-positive cells were quantitated by FACS. For blocking experiments T24 cells, were incubated with human soluble hTfR or transferrin for 30 min at 4° C. prior to the addition of virus. The various viruses are indicated by the hTfR-targeting epitope (B5, B6, etc.). “C” corresponds to Ad5GFP. The data are the means±the SEM and are from three independent experiments.

FIG. 11. hTfR expression and Ad5GFPB6HI- or Ad5GFPB8HI-mediated transduction of human BME cells. Human BME cells (1×10⁵) were transduced with Ad5GFPB6HI, Ad5GFPB8HI, or Ad5GFP (4×10⁸ particles), and GFP-positive cells were quantitated by FACS. For blocking experiments, cells were incubated with transferrin for 30 min at 4° C. prior to the addition of virus. The data are the means±the SEM and are from three independent experiments.

### DETAILED DESCRIPTION OF THE INVENTION

Definitions

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid”, “nucleic acid molecule”, “nucleic acid fragment”, “nucleic acid sequence or segment”, or “polynucleotide” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced. For example, a protein or nucleotide sequence present in an organism (including a virus) that can be isolated from a source in nature and has not been intentionally modified in the laboratory, is naturally occurring.

The term “chimeric” refers to any gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

The terms “protein,” “peptide” and “polypeptide” are used interchangeably herein.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. “Variant nucleotide sequences” may also be synthetically derived nucleotide sequences that encode a native protein that are generated, for example, by using site-directed mutagenesis. “Variant nucleotide sequences” may also be those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” that are one species of “conservatively modified variations.” Every nucleic acid sequence described herein that encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

“Recombinant DNA molecule” is a combination of DNA sequences that are joined together using recombinant DNA technology and procedures used to join together DNA sequences as described, for example, in Sambrook et al., Cold Spring Harbor, N.Y.: Cold Spring Harbor Laboratory Press (1989).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular from which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

“Expression cassette” as used herein means a DNA sequence capable of directing expression of a particular nucleotide sequence in an appropriate host cell, comprising a promoter operably linked to the nucleotide sequence of interest that is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (codon) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide that is translated in conjunction with the polypeptide forming a precursor peptide and is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid or amino acid sequences where the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Amino acid sequence can be operably linked such that a chimeric protein is generated.

“Expression” refers to the transcription and/or translation of an endogenous gene or a transgene in cells. For example, in the case of antisense constructs, expression may refer to the transcription of the antisense DNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to the production of antisense RNA transcripts capable of suppressing the expression of protein from an endogenous gene or a transgene.

“Co-suppression” and “transwitch” each refer to the production of sense RNA transcripts capable of suppressing the expression of identical or substantially similar transgene or endogenous genes (U.S. Pat. No. 5,231,020).

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected. Gene silencing includes virus-induced gene silencing.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule. An example of a cis-acting sequence on the replicon is the viral replication origin.

The terms “trans-acting sequence” and “trans-acting element” refer to DNA or RNA sequences whose function does not require them to be on the same molecule.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al, CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix. See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.”Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6 (log M)+0.41 (%GC) −0.61 (% form) −500/L; where M is the molarity of monovalent cations, %GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5×to 1×SSC at 55 to 60° C.

As used herein, the term “fiber protein” includes variants or biologically active fragments of the adenovirus fiber protein. By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art. For example the fiber protein may be encoded by 100 bases of the native coding sequence. Alternatively, it may be encoded by 210 bases of the native coding sequence.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

It is known that variant polypeptides can be obtained based on substituting certain amino acids for other amino acids in the polypeptide structure in order to modify or improve biological activity. For example, through substitution of alternative amino acids, small conformational changes may be conferred upon a polypeptide that result in increased bioactivity. Alternatively, amino acid substitutions in certain polypeptides may be used to provide residues that may then be linked to other molecules to provide peptide-molecule conjugates that retain sufficient properties of the starting polypeptide to be useful for other purposes.

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity. Accordingly, it is noted that substitutions can be made based on the hydrophilicity assigned to each amino acid. In using either the hydrophilicity index or hydropathic index, which assigns values to each amino acid, it is preferred to conduct substitutions of amino acids where these values are ±2, with ±1 being particularly preferred, and those with in ±0.5 being the most preferred substitutions.

Thus, the genes and nucleotide sequences of the invention include both the naturally occurring sequences as well as mutant forms. Likewise, the polypeptides of the invention encompass both naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (O). In addition, individual substitutions, deletions or additions that alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook et al., Molecular Cloning: A Laboratory Manual (2d ed., Cold Spring Harbor Laboratory Press, Plainview, N.Y.) (1989). See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

By “portion” or “fragment”, as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

As used herein, the term “therapeutic agent” refers to any agent or material that has a beneficial effect on the mammalian recipient. Thus, “therapeutic agent” embraces both therapeutic and prophylactic molecules having nucleic acid or protein components. The mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition that is not attributable to an inborn defect), cancers and prophylactic processes (i.e., prevention of a disease or of an undesired medical condition).

Adenovirus Vectors

Studies using MPS VII mouse models (deficient in the lysosomal enzyme β-glucuronidase) have allowed testing of potential therapies for both the CNS and visceral components of this representative disease. Direct intraparenchymal gene transfer to mouse brain with adenovirus vectors expressing β-glucuronidase allowed for extensive distribution of enzyme and correction of the characteristic storage defect within the brains of β-glucuronidase-deficient mice (Ghodsi et al., Hum. Gene Ther., 9, 2331-2340 (1998); Stein et al., J. Virol., 73, 3424-3429 (1999)). The spread of enzyme beyond sites of transduction resulted from secretion of β-glucuronidase upon overexpression, with uptake and correction by nontransduced cells. Similar results were found with recombinant adeno-associated virus (Sferra et al., Hum. Gene Ther., 11, 507-519 (2000); Skorupa et al., Exp. Neurol., 160, 17-27 (1999); Watson et al., Gene Ther., 5, 1642-1649 (1998)) and lentivirus (Bosch et al., Hum. Gene Ther., 11, 1139-1150 (2000)) vectors expressing β-glucuronidase. Due to the larger size of a primate brain, however, focal gene delivery is unlikely to result in significant amounts of secreted enzyme reaching areas remote from the site of vector injection. An alternative to direct injection into the brain parenchyma for correction of global neurodegenerative disease would be to take advantage of the vasculature of the host. One approach could be to disrupt the tight junctions of the vascular endothelia for direct vector access to the underlying parenchyma. A second could be to transduce the vascular endothelium directly. For β-glucuronidase, which is capable of being secreted basolaterally from vascular endothelium, distribution into the subpial and perivascular spaces (Virchow-Robin spaces) lining the penetrating blood vessels could allow access to the parenchyma since the pia does not form an impermeable barrier.

In earlier studies, it was found that BBB disruption does not result in adequate vector access to parenchymal tissues. Data showed that only several hundred cells could be transduced upon delivery of virus to mannitol-disrupted tight junctions (Doran et al., Neurosurgery, 36, 965-970 (1995)). Rather than delivery of virus through disrupted tight junctions (Doran et al., Neurosurgery, 36, 965-970 (1995); Nilayer et al., Proc. Natl. Acad. Sci. USA, 92, 9829-9833 (1995)), the present invention takes advantage of receptors present on brain vascular endothelium such as the transferrin receptor (TfR). Human TfR (hTfR), a type II membrane protein, has been extensively characterized and consists of two identical 95-kDa subunits linked convalently by two disulfide bonds (Testa et al., Crit. Rev. Oncog., 4, 241-276 (1993)). In vitro, in vivo, and ex vivo studies by Pardridge and others showed that antibody or transferrin conjugates with specificity for the TfR allowed for delivery of substances to brain capillary endothelial cells (Broadwell et al., Exp. Neurol., 142, 47-65 (1996); Friden et al., J. Pharmacol. Exp. Ther., 278, 1491-1498 (1996); Pardridge et al., J. Pharmacol. Exp. Ther., 259, 66-70 (1991); Shin et al., Proc. Natl. Acad. Sci. USA, 92, 2820-2824 (1995)). It was found that adenoviruses with motifs targeting the TfR could also allow for transduction of the brain microcapillary endothelium (BME).

Modification of the virus for targeting to the TfR was accomplished through bifunctional antibodies or by genetically modifying the virus to display a specific TfR binding motif. Douglas et al. and others have reported the feasibility of using bifunctional antibodies for targeting in vitro (Douglas et al., Nat. Biotechnol., 14, 1574-1578 (1996); Harari et al., Gene Ther., 6, 801-807 (1999); Wickham et al., Cancer Immunol. Immunother., 45, 149-151 (1997); Wickham et al., J. Virol., 70, 6831-6838 (1996); Yoon et al., Biochem. Biophys. Res. Commun., 272, 497-504 (2000)). Experiments also showed that adenovirus capsids modified to contain a carboxyl-terminal polylysine tract allowed for improved gene transfer to many cell types via facilitated binding to cell surface heparan sulfate proteoglycans (Bouri et al., Hum. Gene Ther., 10, 1633-1640 (2000); Gonzalez et al., Gene Ther., 6, 314-320 (1999); Staba et al., Cancer Gene Ther., 7, 13-19 (2000); Wickham et al., Nat. Biotechnol., 14, 1570-1573 (1996); Wickham et al., J. Virol., 71, 8221-8229 (1997)). Alteration of the HI loop of fiber to express an RGD motif, a sequence normally found in the penton base of adenovirus type 5 (Ad5), also improved binding to integrin-expressing cells (Reynolds et al., Gene Ther., 6, 1336-1339 (1999)).

Because of its relative simplicity, phage display screening was used to identify epitopes specific to the TfR and then introduced the sequences encoding these peptides directly into the HI loop of Ad5 fiber. Modified recombinant adenoviruses were then tested for their ability to transduce TfR-expressing cell lines and human BME cells.

Several features of adenovirus have made them useful as a transgene delivery vehicles for therapeutic applications, such as facilitating in vivo nucleic acid sequence delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ. In addition, the ability of the adenovirus vector to accomplish in situ tumor transduction has allowed the development of a variety of anti-cancer gene therapy methods for non-disseminated disease. In these methods, vector containment favors tumor cell-specific transduction.

The present invention provides an adenovirus that can be targeted to a desired cell type.

Ligands that may be inserted into the HI loop of the fiber protein include, but are not limited to, tumor necrosis factors (or TNF's) such as, for example, TNF-alpha and TNF-beta; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-1 acid glycoprotein, which binds to the asialoglycoprotein receptor of liver; mannose-containing peptides, which bind to the mannose receptor of macrophages; sialyl-Lewis-X antigen-containing peptides, which bind to the ELAM-1 receptor of activated endothelial cells; CD34 ligand, which binds to the CD34 receptor of hematopoietic progenitor cells; CD40 ligand, which binds to the CD40 receptor of B-lymphocytes; ICAM-1, which binds to the LFA-1 (CD11b/CD18) receptor of lymphocytes, or to the Mac-1 (CD11a/CD 18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; LFA-1, which binds to the ICAM-1 receptor of activated endothelial cells; NGF, which binds to the NGF receptor of neural cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; and the Fv antigen-binding domain of an immunoglobulin.

An adenoviruses of the present invention may be vectors wherein DNA encoding the native or mutated adenoviral fiber protein is operably linked to DNA encoding the ligand. The term “mutated” as used herein means that at least one and no more than 100 amino acid residues of the native adenovirus fiber protein have been changed, or that at least one and no more than 100 amino acid residues of the native adenovirus fiber protein have been deleted from the native adenovirus fiber protein.

The adenoviral vector, in general, also includes DNA encoding at least one therapeutic agent. The term “therapeutic” is used in a generic sense and includes treating agents, prophylactic agents, and replacement agents.

DNA sequences encoding therapeutic agents that may be placed into the adenoviral vector include, but are not limited to, DNA sequences encoding tumor necrosis factor (TNF), such as TNF-alpha; interferons such as Interferon-alpha, Interferon-beta, and Interferon-delta; interleukins such as IL-1, IL-beta, and Interleukins 2 through 14; GM-CSF; adenosine deaminase, or ADA; cellular growth factors, such as lymphokines, which are growth factors for lymphocytes; soluble CD4; Factor VIII; Factor IX; T-cell receptors; the LDL receptor, ApoE, ApoC, ApoAI and other sequences involved in cholesterol transport and metabolism; alpha-1 antitrypsin (alpha-1AT), ornithine transcarbamylase (OTC), CFTR, insulin, viral thymidine kinases, such as the Herpes Simplex Virus thymidine kinase, the cytomegalovirus virus thymidine kinase, and the varicella-zoster virus thymidine kinase; Fe receptors for antigen-binding domains of antibodies, and antisense sequences that inhibit viral replication, such as antisense sequences that inhibit replication of hepatitis B or hepatitis non-A non-B virus.

The DNA sequence encoding at least one therapeutic agent is under the control of a suitable promoter. Suitable promoters that may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; and the ApoAI promoter. It is to be understood, however, that the scope of the present invention is not to be limited to specific foreign nucleic acid sequences or promoters.

The adenoviral vector may, in one embodiment, be an adenoviral vector that includes essentially the complete adenoviral genome. (Shenk, et al., Curr. Top. Microbiol. Immunol, (1984); 111(3):1-39). Alternatively, the adenoviral vector may be a modified adenoviral vector in which at least a portion of the adenoviral genome has been deleted.

In one embodiment, the adenoviral vector comprises an adenoviral 5′ ITR; an adenoviral 3′ ITR; an adenoviral encapsidation signal; at least one DNA sequence encoding a therapeutic agent; and a promoter controlling the at least one DNA sequence encoding a therapeutic agent. The vector is free of the adenoviral E1, E2, E3, and E4 DNA sequences, and the vector is free of DNA sequences encoding adenoviral proteins promoted by the adenoviral major late promoter; i.e., the vector is free of DNA encoding adenoviral structural proteins.

Such vectors may be constructed by removing the adenoviral 5′ ITR, the adenoviral 3′ ITR, and the adenoviral encapsidation signal, from an adenoviral genome by standard techniques. Such components, as well as a promoter (which may be an adenoviral promoter or a non-adenoviral promoter), tripartite leader sequence, poly A signal, and selectable marker, may, by standard techniques, be ligated into a base plasmid or “starter” plasmid such as, for example, pBluescript II KS-(Stratagene), to form an appropriate cloning vector. The cloning vector may include a multiple cloning site to facilitate the insertion of DNA sequence(s) encoding therapeutic agent(s) into the cloning vector. In general, the multiple cloning site includes “rare” restriction enzyme sites; i.e., sites that are found in eukaryotic genes at a frequency of from about one in every 10,000 to about one in every 100,000 base pairs. An appropriate vector is thus formed by cutting the cloning vector by standard techniques at appropriate restriction sites in the multiple cloning site, and then ligating the DNA sequence encoding a therapeutic agent(s) into the cloning vector.

The vector may then be packaged into infectious viral particles using a helper adenovirus that provides the necessary encapsidation materials. Preferably the helper virus has a defective encapsidation signal in order that the helper virus will not encapsidate itself. An example of an encapsidation defective helper virus that may be employed is described in Grable, et al., J. Virol., 66:723-731 (1992).

In another embodiment, the vector comprises an adenoviral 5′ ITR; an adenoviral 3′ ITR; an adenoviral encapsidation signal; at least one DNA sequence encoding a therapeutic agent(s); and a promoter controlling the DNA sequence(s) encoding a therapeutic agent(s). The vector is free of at least the majority of adenoviral E1 and E3 DNA sequences, but is not free of all of the E2 and E4 DNA sequences, and DNA sequences encoding adenoviral proteins promoted by the adenoviral major late promoter. In one embodiment, the vector is also free of at least a portion of at least one DNA sequence selected from the group consisting of the E2 and E4 DNA sequences. In another embodiment, the vector is free of at least the majority of the adenoviral E1 and E3 DNA sequences, and is free of one of the E2 and E4 DNA sequences, and is free of a portion of the other of the E2 and E4 DNA sequences.

In yet another embodiment, the vector is free of at least the majority of the E1 and E3 DNA sequences, is free of at least a portion of at least one DNA sequence selected from the group consisting of the E2 and E4 DNA sequences, and is free of DNA sequences encoding adenoviral proteins promoted by the adenoviral major late promoter.

Such a vector, in a preferred embodiment, is constructed first by constructing, according to standard techniques, a shuttle plasmid that contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a tripartite leader sequence, a multiple-cloning site (which may be as hereinabove described); a poly-A signal; and a DNA segment that corresponds to a segment of the adenoviral genome. Such DNA segment serves as a substrate for homologous recombination with a modified or mutated adenovirus, and such sequence may encompass, for example, a segment of the adenovirus 5 (Ad5) genome no longer than from base 3329 to base 6246 of the genome. The plasmid may also include a selectable marker and an origin of replication. The origin of replication may be a bacterial origin of replication. A desired DNA sequence encoding a therapeutic agent may then be inserted into the multiple cloning site. Homologous recombination is then effected with a modified or mutated adenovirus in which at least the majority of the E1 and E3 adenoviral DNA sequences have been deleted. Such homologous recombination may be effected through co-transfection of the shuttle plasmid and the modified adenovirus into a helper cell line, such as 293 cells, by CaPO₄ precipitation. Upon such homologous recombination, a recombinant adenoviral vector is formed that includes DNA sequences derived from the shuttle plasmid between the Not I site and the homologous recombination fragment, and DNA derived from the E1 and E3 deleted adenovirus between the homologous recombination fragment and the 3′ ITR.

Through such homologous recombination, a vector is formed that includes an adenoviral 5′ ITR, an adenoviral encapsidation signal; an E1a enhancer sequence; a promoter; a tripartite leader sequence; at least one DNA sequence encoding a therapeutic agent; a poly A signal; adenoviral DNA free of at least the majority of the E1 and E3 adenoviral DNA sequences; and an adenoviral 3′ ITR. This vector may then be transfected into a helper cell line, such as the 293 helper cell line, which will include the E1a and E1b DNA sequences, which are necessary for viral replication, and to generate infectious viral particles.

The vector is transfected into an appropriate cell line for the generation of infectious viral particles wherein the adenovirus fiber includes a ligand that is specific for a receptor located on a desired cell type. Transfection may take place by electroporation, calcium phosphate precipitation, microinjection, or through proteoliposomes.

Examples of appropriate cell lines include, but are not limited to, HeLa cells or 293 (embryonic kidney epithelial) cells. The infectious viral particles may then be administered in vivo to a host. The host may be an animal host, including mammalian hosts and human hosts. Such viral particles are “targetable,” i.e., the viral particles, upon administration to the host, will bind to and infect a desired target cell or tissue, and thereby delivering DNA encoding a therapeutic agent to the desired target cell or tissue. The particular target cell or tissue to which the particles are targeted is dependent upon the ligand with which the particle is engineered.

The vector, which consists of an infectious adenovirus particle having a modified fiber protein, is administered in an amount effective to provide a therapeutic effect in a host. In one embodiment, the vector may be administered in an amount of from 1 plaque forming unit to about 10¹⁴ plaque forming units, preferably from about 10⁶ plaque forming units to about 10¹³ plaque forming units. The host may be a human or non-human animal host.

Preferably, the infectious vector particles are administered systemically, such as, for example, by intravenous administration (such as, for example, portal vein injection or peripheral vein injection), intramuscular administration, intraperitoneal administration, or intranasal administration.

The vector particles may be administered in combination with a pharmaceutically acceptable carrier suitable for administration to a patient. The carrier may be a liquid carrier (for example, a saline solution), or a solid carrier, such as, for example, microcarrier beads.

The vector particles, which include a fiber that is engineered with a ligand that is specific for a receptor located on a desired cell type, travel directly to the desired cells or tissues upon the in vivo administration of such vector particles to a host, and whereby such vector particles infect the desired cell or tissues.

Cells that may be infected by the infectious viral particles include, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells; activated endothelial cells; epithelial cells; keratinocytes; stem cells; hepatocytes, including hepatocyte precursor cells; fibroblasts; mesenchymal cells; mesothelial cells; parenchymal cells; vascular smooth muscle cells; brain cells and other neural cells; gut enterocytes; gut stem cells; and myoblasts. The cell that is “targeted” or infected or transduced with the infectious viral particles is dependent upon the ligand with which the infectious viral particle has been engineered.

In one embodiment, the infectious viral particles may be targeted to blood cells, whereby such vector particles infect the blood cells with a sequence that directly or indirectly enhances the therapeutic effects of the blood cells. The sequence carried by the blood cells can be any sequence that allows the blood cells to exert a therapeutic effect that it would not ordinarily have, such as a sequence encoding a clotting factor useful in the treatment of hemophilia. The sequence can encode one or more products having therapeutic effects. Examples of suitable sequences include those that encode cytokines such as TNF, interleukins (interleukins 1-14), interferons (alpha, beta, delta-interferons), T-cell receptor proteins and Fc receptors for antigen-binding domains of antibodies, such as immunoglobulins. Other examples of suitable sequences include sequences encoding soluble CD4 that is used in the treatment of AIDS and sequences encoding alpha-antitrypsin, which is useful in the treatment of emphysema caused by alpha-antitrypsin deficiency.

The infected cells are useful in the treatment of a variety of diseases including but not limited to adenosine deaminase deficiency, sickle cell anemia, thalassemia, hemophilia, diabetes, alpha-antitrypsin deficiency, brain disorders such as Alzheimer's disease, phenylketonuria and other illnesses such as growth disorders and heart diseases, for example, those caused by alterations in the way cholesterol is metabolized and defects of the immune system.

In another embodiment, the vector particles may be targeted to and infect liver cells, and such vector particles may include sequence(s) encoding polypeptides or proteins that are useful in prevention and therapy of an acquired or an inherited defect in hepatocyte (liver) function. For example, they can be used to correct an inherited deficiency of the low density lipoprotein (LDL) receptor, and/or to correct an inherited deficiency of ornithine transcarbamylase (OTC), which results in congenital hyperammonemia.

In another embodiment, the viral particles may be targeted to liver cells, whereby the viral particles include a sequence encoding a therapeutic agent employed to treat acquired infectious diseases, such as diseases resulting from viral infection. For example, the infectious viral particles may be employed to treat viral hepatitis, particularly hepatitis B or non-A non-B hepatitis. For example, an infectious viral particle containing a sequence encoding an anti-sense sequence could be employed to infect liver cells to inhibit viral replication. In this case, the infectious viral particle, which includes a vector including a structural hepatitis sequence in the reverse or opposite orientation, would be introduced into liver cells, resulting in production in the infected liver cells of an anti-sense sequence capable of inactivating the hepatitis virus or its RNA transcripts. Alternatively, the liver cells may be infected with an infectious viral particle including a vector that includes a sequence that encodes a protein, such as, for example, alpha-interferon, which may confer resistance to the hepatitis virus.

Alternatively, the vector particles including the modified adenovirus fiber and a sequence encoding a desired protein or therapeutic agent may be employed to infect a desired cell line in vitro, whereby the infected cells produce a desired protein or therapeutic agent in vitro.

Ad30 Does Not Mediate Transduction via CAR

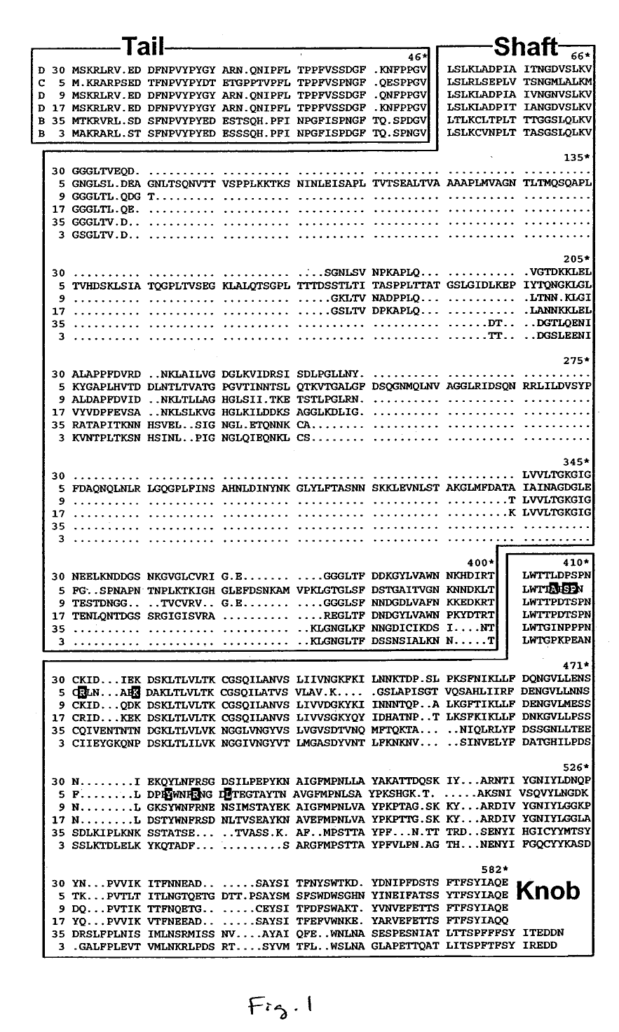

The Ad30 fiber gene. A study of the properties of the Ad30 fiber necessitated cloning and sequencing the gene. Ad30 genomic DNA was isolated from wild-type particles, which had been propagated in HEK 293 cells and purified. The fiber gene was amplified by means of degenerate primers based on the known sequences of other D serotype fibers. As sequence data were acquired further specific primers were designed and employed until the entire sequence of the Ad30 fiber gene was obtained. The amino acid sequence of Ad30 is shown in FIG. 1. Ad30 fiber is very similar to other known D-serotype fibers, Ad9 and 17, and less so to Ad3 or Ad35 fibers. The most divergence was found between Ad30 and Ad5 fibers.

At the protein level, Ad30 fiber shares 25% overall identity with Ad5 fiber. When analyzed according to regions within the fiber protein 61%, 11% and 45% identity was found in the regions of tail, shaft and knob respectively. The 11% identity in the shaft region results primarily from differences in the shaft length between the two fibers. Ad30 fiber is 209 amino acids shorter than Ad5. In contrast, 98%, 55% and 66% identities were found between the Ad30 and Ad9 tail, shaft, and knob regions, respectively. Ad30 fiber was 66% identical to the Ad9 fiber.

Recent work investigated the binding of recombinant fibers to immobilized CAR, and found specific residues important in CAR binding. Of the 4 residues shown to be critical for CAR binding (Ser₄₀₈, Pro₄₀₉, Tyr₄₇₇ and Leu₄₈₅), all but one (Leu₄₈₅) were conserved in Ad30 fiber (Kirby, I., et al. 2000. J. Virol. 74(6):2804-2813; Roelvink, P. et al., 1999 Science 286:1568-1571). Of the 3 amino acid residues that may peripherally effect CAR binding (Ala₄₀₆, Arg₄₁₂ and Arg₄₈₁), only one (Arg₄₈₁) has been conserved. These differences are illustrated in FIG. 1.

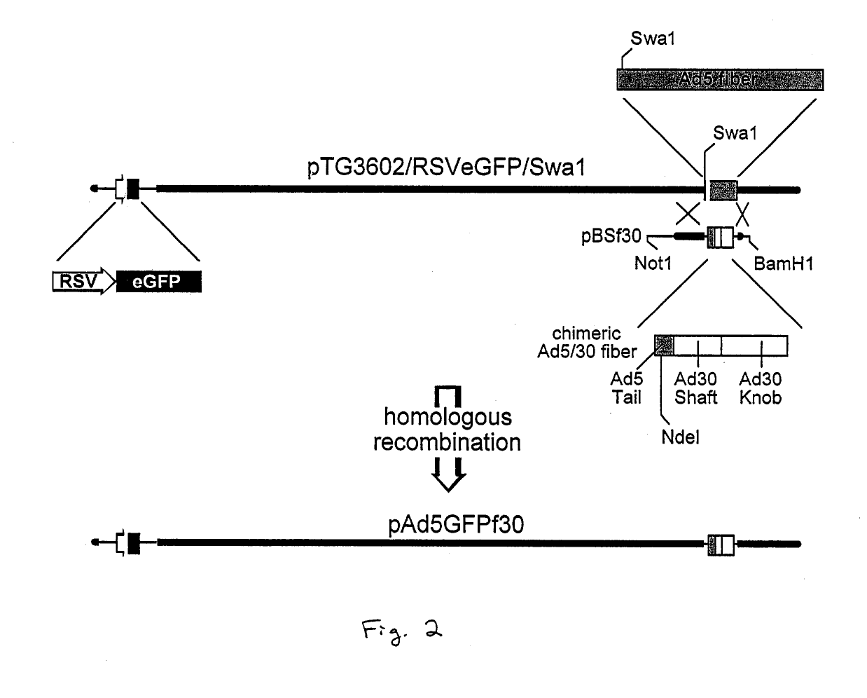

Chimeric fiber. To test the properties of the Ad30 fiber independent of the Ad30 virion, a chimeric virus was generated (Ad5GFPf30) by homologous recombination in E. coli. (Anderson, R. D. et al 2000. Gene Ther. 7(12):1034-1038). The Ad30 fiber gene was generated by overlapping PCR and, similar to earlier work with Ad17 fiber (Chillon, M., et al. 1999 J. Virol. 73(3):2537-2540), was designed to retain the Ad5 tail, and replace Ad5 shaft and knob sequences with those from Ad30. The fiber fragment was cloned into the Ad5 backbone in place of the endogenous Ad5 fiber as depicted in FIG. 2. Sequence analysis of the plasmid pAd5GFPf30 confirmed the presence of the chimeric fiber. pAd5GFPf30 was linearized by PacI and transfected into HEK 293 cells to generate virus. Western blot analysis of purified particles verified the expression of the shortened, chimeric fiber (FIG. 3). Ad5GFPf30-induced cytopathic effect (CPE) was delayed relative to Ad5GFP (45 vs. 30 h), and viral yields (numbers of particles and infectious units) were lower by two to three-fold. The titer of Ad5GFPB30 was 4×10⁹ PFU/ml, and that for Ad5GFP was 1×10⁰¹ PFU/ml.

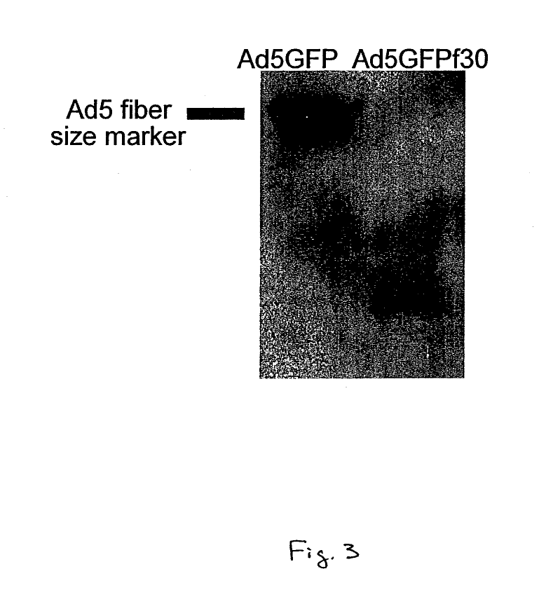

Ad5 has been shown to infect cells via CAR. To assess the potential use of the CAR receptor by the hybrid virus, CHO cells were used. This cell type has been shown to express little if any endogenous receptor, but when made to express CAR, can direct Ad5-mediated gene transfer. Ad5GFP infected 1% of control-virus-transfected CHO cells (FIG. 4). Similar results were seen with Ad5GFPf30 (FIG. 4). To convert to a CAR-expressing phenotype, CHO cells were incubated with an AdhCAR:CaPᵢ co-precipitant for thirty minutes at 37° C., resulting in 96% of cells being positive for CAR expression as determined by FACS. CAR expression resulted in significant increases in Ad5GFP-mediated gene transfer from 1% to 36% of GFP-positive cells. In contrast, the introduction of CAR had no appreciable impact on the level of infection efficiency of Ad5GFPf30, which remained at ≦1% (FIG. 4).

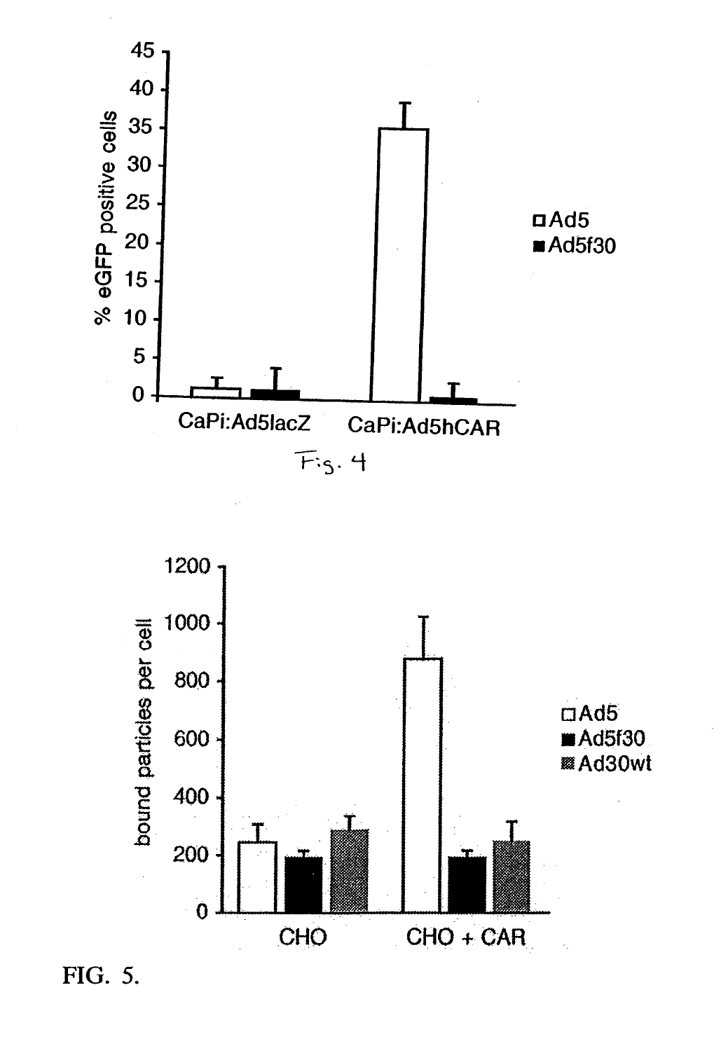

Binding studies were done to determine if the lack of transduction of CAR-expressing CHO cells was due to limited binding or to inhibited entry. Ad5GFP, Ad5GFPf30 and wild-type Ad30 viruses were labeled with [methyl-3H] thymidine and applied to CHO cells, or CHO cells that had been transfected by AdhCAR-CaPᵢ co-precipitant as described above. Cells were incubated with equivalent numbers of labeled particles, and unbound virus was removed. While the presence of CAR resulted in a 3.6-fold increase in bound Ad5GFP particles, CAR expression had no effect on Ad5GFPf30 or wild-type Ad30 binding (FIG. 5). These data indicate that Ad5GFPf30 and wildtype Ad30 does not bind CAR.

Receptor Targeting Motifs

The efficiency of adenovirus infection depends on the level of the coxackie adenovirus receptor (CAR) expression. However, the tropism of Ad5 can be modified using genetic methods (Reynolds, Gene Ther. 6:1336-1339 (1999); Wickham et al Nat. Biotechnol. 14:1570-1573 (1996)). As a first step toward extending the tropism of Ad5 to BME for use in CNS gene therapy, a nonapeptide phage display library was screened against the extracellular domain of hTfR (Testa, Crit. Rv. Oncog. 4:241-276 (1993)), a receptor found at high density on the BME. Enzyme-linked immunosorbent assay (ELISA) plates were coated with soluble hTfR, and bound phage were eluted by a low-pH buffer. Eluted phage were amplified in E. coli K91 and subsequently screened again for binding to the immobilized hTfR. In separate experiments, bound phage were eluted with purified transferrin holoenzyme. Three rounds of screening-amplification were done for each type of elution. From two independent experiments, a total of 43 clones were isolated, most from acid elution. Sequencing of isolated phage revealed the peptide motifs AkxxK/Rx (SEQ ID NO:7), KxKxPK/R (SEQ ID NO:8), or KxK in 31 of the clones (Table 1). Some clones were isolated more than once, arising from independent experiments and different elution parameters. GHKVKRPKG (SEQ ID NO:9) and IEAYAKKRK (SEQ ID NO:10) motifs were isolated 10 and 7 times, respectively. The peptide sequence KDKIKMDKK (SEQ ID NO: 11) was present in four clones, while KNKIPKSPK (SEQ ID NO:12) was isolated twice. Only several peptides contained amino acid arrays identical to regions of human transferrin (Table 1), indicating that most motifs were likely conformational, rather than linear epitopes.

TABLE 1

Displayed Phage targeted to the TfR^{a}

| | |
|---|---|
| Group | Sequence |

| 1 | LQAKKKRPK | (SEQ ID NO:13) |
|---|---|---|
| | VIAKIKKPK | (SEQ ID NO:14) |
| | AIAKKHKWN | (SEQ ID NO:15) |
| | VEAKGHKKK | (SEQ ID NO:16) |
| | IEAYAKKRK^{c} | (SEQ ID NO:10) |
| | GHKAKGPRK^{b} | (SEQ ID NO:17) |
| 2 | LQAKKKRPK | (SEQ ID NO:13) |
| | VIAKIKKPK | (SEQ ID NO:14) |
| | KWKTPKVRV | (SEQ ID NO:18) |
| | KNKIPKSPK^{c} | (SEQ ID NO:12) |
| | GHKAKGPRK^{b} | (SEQ ID NO:17) |
| | GHKVKRPKG^{c} | (SEQ ID NO:9) |
| | GKGPKWMR^{d, e} | (SEQ ID NO:19) |
| 3 | VEAKGHKKK | (SEQ ID NO:16) |
| | AIAKKHKWN | (SEQ ID NO:15) |
| | LQAKKKRPK | (SEQ ID NO:13) |
| | VIAKIKKPK | (SEQ ID NO:14) |
| | KWKTPKVRV | (SEQ ID NO:18) |
| | KWKLHGHIK | (SEQ ID NO:20) |
| | KNKIPKSPK^{c} | (SEQ ID NO:12) |
| | GHKAKGPRK^{b} | (SEQ ID NO:17) |
| | IEAYAKKRK^{c} | (SEQ ID NO:10) |
| | GHKVKRPKG^{c} | (SEQ ID NO:9) |
| | KDKIKMDKK^{b, e, f} | (SEQ ID NO:11) |
| ^{a}Unless otherwise annotated, all epitopes were identified by acid elution. | | |
| Underlined sequences indicate amino acids found in human transferrin. | | |

^{b}Epitope identified using ligand elution parameters.
^{c}Epitope identified using both ligand and acid elution parameters.
^{d}This epitope contained only 8 amino acids.
^{e}Similar to KGKK in transferrin.
^{f}Similar to KDKSK in transferrin.

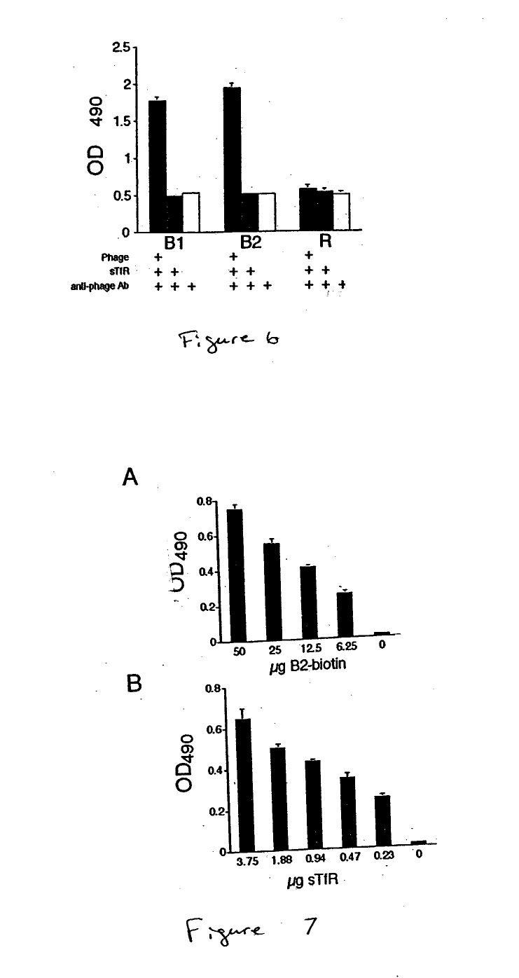

To test the specificity of peptide binding for human TfR, two motifs were randomly chosen: IEAYAKKRK (B1; SEQ ID NO: 10) and GHKVKRPKG (B2; SEQ ID NO:9). First, phage expressing the B1 or B2 peptide and randomly isolated control phage (R) were subjected to large-scale amplification, followed by incubation with immobilized hTfR. Both B 1 and B2 bound hTfR, in contrast to control phage (FIG. 6). Binding of the B2 peptide motif to hTfR, independent of the phage sequences, was also tested. Biotinylated B2 was synthesized and incubated with immobilized hTfR using standard ELISA-based assays. The data in FIGS. 7A and 7B show that peptide B2 alone bound to hTfR in a dose-dependent manner.

Characterization of Recombinant Fibers

Motifs in phage may or may not be indicative of the same sequence in the context of Ad5 fiber. Moreover, the hTfR-targeting motifs could have deleterious effects on fiber trimerization inhibiting their assembly and in turn impairing virus capsid maturation. To test if the identified nonapeptides retained their ability to bind hTfR in the context of fiber, immunoprecipitation experiments were performed. First, motifs were cloned into the HI loop and the resultant fibers were expressed in a mammalian expression system. Lysates containing the modified fibers were then incubated with hTfR, and the complex was immunoprecipitated with anti-hTfR antibodies. Western blot analysis for fiber using an antibody to the amino-terminal region indicated that the hTfR-targeting motifs, when placed in the HI loop of fiber, maintained their ability to bind to hTfR. Wild-type fiber, though present at high levels in the expressed lysate, was not pulled down with anti-hTfR antibodies, demonstrating specificity of the epitopes for the hTfR.

Lysates containing expressed, modified fibers were also used to determine the effects of the motifs on fiber trimerization. Other researchers have shown that trimerization can be disrupted by modifications at the carboxy terminus (Hong et al., J. Virl. 70:7071-7078 (1996)). The present studies showed that the motifs did not inhibit fiber trimerization. Similar results were seen for the other motifs.

HI Loop-Modified Viruses and Gene Transfer to Receptor-Expressing Cells

The generation of multiple recombinant adenoviral vectors differing only in the HI loop could be best accomplished using the E. coli recombination system developed by Chartier et al. (J. Virol. 70:4805-4810 (1996)). For the present purposes, two key plasmids were developed. The first was pBS/B2HI. This plasmid contained Ad5 fiber sequences with a unique SpeI site at the 3′ end of the fiber gene. This site, in combination with an internal SphI site, allowed the subsequent introduction of all other HI modifications (pBS/B1HI, pBS/B3HI, etc.). It is important to note that pBS/B2HI will also allow the efficient introduction of any fiber modification. The fiber sequences contained more than 1.0 kb of flanking sequence for increased efficiency of recombination in E. coli.

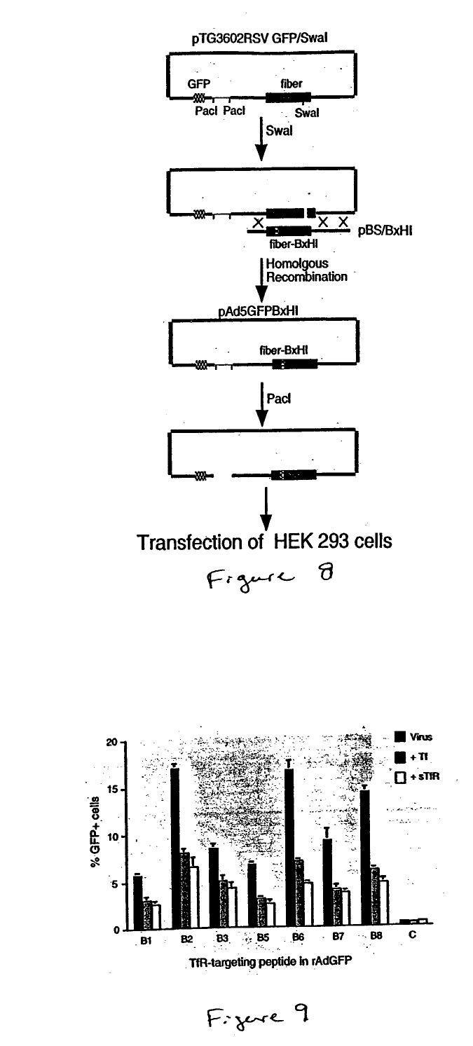

A plasmid containing a full-length adenovirus genome with a green fluorescent protein (GFP) expression cassette in the E1 region and a unique SwaI site in fiber was also generated. The SwaI site facilitated recombination with the pBS/BxHI plasmids (FIG. 8). The resultant plasmids were digested with PacI and transfected into HEK 293 cells. Considerable variability was noted in the length of time from transfection to CPE and also the ability to further amplify the viruses once the initial lysates were harvested. Of the ten peptides cloned into the fiber of Ad5, only seven could be amplified and purified to concentrations adequate for further testing. These results indicate that amplification of the HI loop adenoviruses required retention of CAR binding for adequate growth in HEK 293 cells. Thus, the viruses have expanded rather than targeted tropism.

Peptide-modified recombinant adenoviruses were tested for their ability to transduce a CHO cell line previously transfected with recombinant hTfR. In this cell line, the endogenous CHO TfR has a 51-amino-acid deletion in the cytoplasmic domain leading to a nonfunctional receptor (Recht et al., Cancer Immunol. Immunother. 42:357-361 (1996)). Recombinant adenoviruses containing motifs B1 to B3 and B5 to B8 (Ad5GFPB1HI, Ad5GFPB2HI, etc.) facilitated gene transfer 11- to 34-fold over Ad5GFP (FIG. 9). In all cases, infection was inhibited by preincubating the cells with human transferrin, suggesting that gene transfer with TfR motif-modified adenoviruses occurred in part through specific binding to the hTfR. Preincubation of cells with soluble hTfR corroborated these results (FIG. 9).

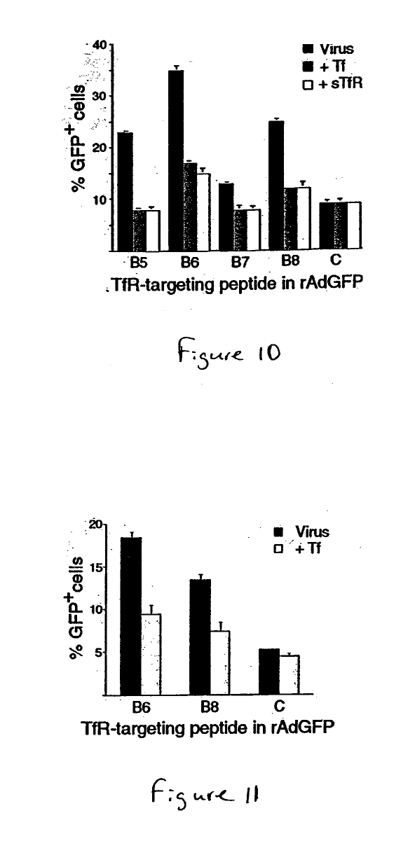

Ad5GFPB5HI through Ad5GFPB8HI were further tested on T24 cells, a cell line that has high endogenous levels of hTfR and undetectable levels of CAR as assessed by reverse transcription-PCR and FACS analysis. All motifs directed significant increases in gene transfer to T24 cells (FIG. 10). Again, B6 and B8 epitopes were best. Ad5GFPB6HI and Ad5GFPB8HI allowed for transduction of 25 and 15% of cells (subtracting the background), respectively, a 3.9- or 2.8-fold increase over the control virus. Further, this increase could be abrogated by the prior addition of transferrin or purified soluble hTfR (FIG. 10).

Human BME cells express a high level of the TfR. Initial studies showed that these cells are poorly transduced by recombinant adenoviruses expressing native Ad5 fiber sequences (FIG. 11). However, when Ad5GFPB6HI and Ad5GFPB8HI were tested, gene transfer to human BME improved 3.5- and 2.5-fold, respectively. Again, transduction could be inhibited by preincubation with transferrin, indicating that the recombinant viruses were binding and entering BME cells via the targeted motif.

Methods of Generating Adenoviral Vectors

Recombinant adenoviruses are useful vectors for basic research and for clinical applications. When used in delineating protein function, vectors that contain a given transgene with mutations or alterations to the coding sequence are compared at the same time. Adenoviruses can be made by standard transfection of a shuttle plasmid and viral DNA backbone into HEK 293 cells. Homologous recombination occurs in vivo, and recombinant virus can be isolated and propagated. The major drawback of this technique is that the starting viral DNA backbone, restricted of E1 containing sequences, must be 100% free of full-length Ad DNA. Otherwise, varying amounts of wild-type virus are also propagated. Alternatively, adenoviruses can be made via the streamlined method set forth in U.S. patent application Ser. No. 09/521,524 and in Anderson, et al., (2000) Gene Ther. 7(12):1034-1038.

Methods of Treating Genetic Disease or Cancer

The present invention provides methods of treating a genetic disease or cancer in a mammal by administering a polynucleotide, polypeptide, expression vector, viral particle or cell. For the gene therapy methods, a person having ordinary skill in the art of molecular biology and gene therapy would be able to determine, without undue experimentation, the appropriate dosages and routes of administration of the a polynucleotide, polypeptide, expression vector, viral particle or cell used in the novel methods of the present invention.

The instant invention provides a cell expression system for expressing exogenous genetic material in a mammalian recipient. The expression system, also referred to as a “genetically modified cell,” comprises a cell and an expression vector for expressing the exogenous genetic material. The genetically modified cells are suitable for administration to a mammalian recipient, where they replace the endogenous cells of the recipient. Thus, the preferred genetically modified cells are non-immortalized and are non-tumorogenic.

According to one embodiment, the cells are transformed or otherwise genetically modified ex vivo. The cells are isolated from a mammal (such as a human), transformed (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transformed or otherwise genetically modified in vivo. The cells from the mammalian recipient, are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into anti-sense RNA, as well as a “heterologous sequence” (i.e., a sequence encoding a protein that is not expressed or is expressed at biologically insignificant levels in a naturally-occurring cell of the same type). To illustrate, a synthetic or natural sequence encoding human erythropoietin (EPO) would be considered “exogenous genetic material” with respect to human peritoneal mesothelial cells since the latter cells do not naturally express EPO; similarly, a human interleukin-1 gene inserted into a peritoneal mesothelial cell would also be an exogenous gene to that cell since peritoneal mesothelial cells do not naturally express interleukin-1 at biologically significant levels. Still another example of “exogenous genetic material” is the introduction of only part of a genetic sequence to create a recombinant sequence, such as combining an inducible promoter with an endogenous coding sequence via homologous recombination.

In the certain embodiments, the mammalian recipient has a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition that is not attributable to an inborn defect), cancers and prophylactic processes (i.e., prevention of a disease or of an undesired medical condition). Accordingly, as used herein, the term “therapeutic agent” refers to any agent or material that has a beneficial effect on the mammalian recipient. Thus, “therapeutic agent” embraces both therapeutic and prophylactic molecules having nucleic acid (e.g., antisense RNA) and/or protein components.

A number of diseases caused by single-gene defects have been identified (Roemer, K. and Friedmann, T., Eur J. Biochem. 208:211-225 (1992); Miller, A. D., Nature 357:455-460 (1992); Larrick, J. W. and Burck, K. L. Gene Therapy. Application of Molecular Biology, Elsevier, N.Y., (1991) and references contained therein). Examples of these diseases, and the therapeutic agents for treating the exemplary diseases, are provided in Table 2 below.

TABLE 2
Therapeutic Agents for Treating Diseases Involving Single-Gene Defects*

| Disease | Therapeutic Agent |
|---|---|
| Immunodeficiency | Adenosine deaminase |
| | Purine nucleoside phosphorylase |
| Hypercholesterolaemia | LDL receptor |
| Haemophilia A | Factor VIII |
| Haemophilia B | Factor IX |
| Gaucher's disease | Glucocerebrosidase |
| Mucopolysaccharidosis | β-glucuronidase |
| Emphysema | α₁-antitrypsin |
| Cystic fibrosis | Cystic fibrosis trans-membrane |
| | regulator |
| Phenylketonuria | Phenylalanine hydroxylase |
| Hyperammonaemia | Ornithine transcarbamylase |
| Citrullinaemia | Arginosuccinate synthetase |
| Muscular dystrophy | Dystrophin |
| Thalassaemia | β-globin |
| Sickle cell anaemia | α-globin |
| Leukocyte adhesion deficiency | CD-18 |
| von Willebrand's disease | von Willebrand Factor |

*see Roemer, K. and Friedmann, T., Eur J. Biochem. 208: 211-225 (1992) and Miller, A. D., 1992, Nature 357: 455-460 and references contained therein

As used herein, “acquired pathology” refers to a disease or syndrome manifested by an abnormal physiological, biochemical, cellular, structural, or molecular biological state. Exemplary acquired pathologies, and the therapeutic agents for treating the exemplary pathologies, are provided in Table 3 below.

TABLE 3
Therapeutic Agents for Acquired Pathologies

Pathologies Associated with Peritoneal Dialysis

| | |
|---|---|
| Anemia | Erythropoietin |
| Peritoneal sclerosis | Fibrinolytic agents (e.g., tissue plasminogen |
| | activator (t-PA), or single chain urokinase |
| | plasminogen activator (scu-PA) |
| Peritonitis | Anti-oxidants (e.g., Superoxide Dismutase, |
| | Catalase) |
| Uremia | Urease |

Other Conditions

| | |
|---|---|
| Septic Shock | Anti-thrombotic agents (e.g., elastase-resistant |
| | form of thrombomodulin (TM)) |
| Diabetes mellitus | Insulin |
| Pituitary Dwarfism | Human growth hormone |
| Thrombosis | Hirudin, secreted form of TM |
| Post-Surgical Adhesions | Anti-thrombotic agents (e.g., thrombomodulin, |
| | hirudin), Fibrinolytic agents (e.g., TPA, scu- |
| | PA), Surfactants |
| AIDS | CD-4 |

The condition amenable to gene replacement therapy alternatively can be a genetic disorder or an acquired pathology that is manifested by abnormal cell proliferation, e.g., cancers. According to this embodiment, the instant invention is useful for delivering a therapeutic agent having anti-neoplastic activity (i.e., the ability to prevent or inhibit the development, maturation or spread of abnormally growing cells), to primary or metastasized tumors, (e.g., ovarian carcinoma, mesothelioma, colon carcinoma). Therapeutic agents for treating these and other cancers include, for example, the anti-neoplastic agents provided in Table 4.

TABLE 4
Therapeutic Agents for Treating Cancers*

| Defective Gene | Therapeutic Agent |
|---|---|
| Oncogenes | corresponding normal genes, oncogene antisense |
| | RNA, Mutated Tumor-Suppressor genes, Normal |
| | Tumor-Suppressor (e.g., p53) |
| Unidentified defect | cytokines, the interferons, tumor necrosis factor, the |
| | interleukins. |

*see Roemer, K. and Friedmann, T., 1992, supra., and references contained therein.

Delivery of a therapeutic agent by a genetically modified cell is not limited to delivery to a particular location in the body in which the genetically modified cells would normally reside. For example, it is possible that a therapeutic agent secreted by a genetically modified cell within a coelomic cavity could reach the lymphatic network draining that coelomic cavity. Accordingly, the genetically modified cells of the invention are useful for delivering a therapeutic agent, such as an anti-neoplastic agent, to various parts of the body.

Alternatively, the condition amenable to gene replacement therapy is a prophylactic process, i.e., a process for preventing disease or an undesired medical condition. Thus, the instant invention embraces a cell expression system for delivering a therapeutic agent that has a prophylactic function (i.e., a prophylactic agent) to the mammalian recipient. Such therapeutic agents (with the disease or undesired medical condition they prevent appearing in parentheses) include: estrogen/progesterone (pregnancy); thyroxine (hypothyroidsm); and agents that stimulate, e.g., gamma-interferon, or supplement, e.g., antibodies, the immune system response (diseases associated with deficiencies of the immune system).

In summary, the term “therapeutic agent” includes, but is not limited to, the agents listed in Tables 2-4, as well as their variants or functional equivalents. As used herein, the term “functional equivalent” refers to a molecule (e.g., a peptide or protein) that has the same or an improved beneficial effect on the mammalian recipient as the therapeutic agent of which is it deemed a functional equivalent. Accordingly, the instant invention embraces therapeutic agents encoded by naturally-occurring DNAs, as well as by non-naturally-occurring DNAs that encode the same protein as encoded by the naturally-occurring DNA.

The above-disclosed therapeutic agents and conditions amenable to gene replacement therapy are merely illustrative and are not intended to limit the scope of the instant invention. The selection of a suitable therapeutic agent for treating a known condition is deemed to be within the scope of one of ordinary skill of the art without undue experimentation.

Methods for Introducing Genetic Material into Cells

The exogenous genetic material (e.g., a cDNA encoding one or more therapeutic proteins) is introduced into the cell ex vivo or in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation (Methods in Molecular Biology, Vol. 7, Gene Transfer and Expression Protocols, Ed. E. J. Murray, Humana Press (1991)); DEAE-dextran; electroporation; cationic liposome-mediated transfection; and tungsten particle-faciliated microparticle bombardment (Johnston, S. A., Nature 346:776-777 (1990)). Strontium phosphate DNA co-precipitation (Brash D. E. et al. Molec. Cell. Biol. 7:2031-2034 (1987) is an alternative transfection method.

In contrast, “transduction of cells” refers to the process of transferring nucleic acid into a cell using virus. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. An expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the β-actin promoter (Lai et al., Proc. Natl. Acad. Sci. USA 86: 10006-10010 (1989)), and other constitutive promoters known to those of skill in the art. In addition, many viral promoters function constitutively in eucaryotic cells. These include the early and late promoters of SV40, the long terminal repeats (LTRs) of Moloney Leukemia Virus and other retroviruses, and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) that stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters that control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may also include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The therapeutic agent can be targeted for delivery to an extracellular, intracellular or membrane location. If it is desirable for the gene product to be secreted from the cells, the expression vector is designed to include an appropriate secretion “signal” sequence for secreting the therapeutic gene product from the cell to the extracellular milieu. If it is desirable for the gene product to be retained within the cell, this secretion signal sequence is omitted. In a similar manner, the expression vector can be constructed to include “retention” signal sequences for anchoring the therapeutic agent within the cell plasma membrane. For example, all membrane proteins have hydrophobic transmembrane regions that stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

The following discussion is directed to various utilities of the instant invention. For example, the instant invention has utility as an expression system suitable for detoxifying intra- and/or extracellular toxins in situ. By attaching or omitting the appropriate signal sequence to a gene encoding a therapeutic agent capable of detoxifying a toxin, the therapeutic agent can be targeted for delivery to the extracellular milieu, to the cell plasma membrane or to an intracellular location. In one embodiment, the exogenous genetic material containing a gene encoding an intracellular detoxifying therapeutic agent, further includes sequences encoding surface receptors for facilitating transport of extracellular toxins into the cell where they can be detoxified intracellularly by the therapeutic agent. Alternatively, the cells can be genetically modified to express the detoxifying therapeutic agent anchored within the cell plasma membrane such that the active portion extends into the extracellular milieu. The active portion of the membrane-bound therapeutic agent detoxifies toxins that are present in the extracellular milieu.

In addition to the above-described therapeutic agents, some of which are targeted for intracellular retention, the instant invention also embraces agents intended for delivery to the extracellular milieu and/or agents intended to be anchored in the cell plasma membrane.

The selection and optimization of a particular expression vector for expressing a specific gene product in an isolated cell is accomplished by obtaining the coding sequence, such as with one or more appropriate control regions (e.g., promoter, insertion sequence); preparing a vector construct comprising the vector into which is inserted the coding sequence; transfecting or transducing cultured cells in vitro with the vector construct; and determining whether the gene product is present in the cultured cells.

In the present invention the adenovirus is used as an expression vector for transformation of cells. The adenovirus is frequently responsible for respiratory tract infections in humans and thus appears to have an avidity for the epithelium of the respiratory tract (Straus, S., The Adenovirus, H. S. Ginsberg, Editor, Plenum Press, New York, P. 451-496 (1984)). Moreover, the adenovirus is infective in a wide range of cell types, including, for example, muscle and endothelial cells (Larrick, J. W. and Burck, K. L., Gene Therapy. Application of Molecular Biology, Elsevier Science Publishing Co., Inc., New York, p. 71-104 (1991)). The adenovirus also has been used as an expression vector in muscle cells in vivo (Quantin, B., et al., Proc. Natl. Acad. Sci. USA 89:2581-2584 (1992)).

The adenovirus genome is adaptable for use as an expression vector for gene therapy, i.e., by removing the genetic information that controls production of the virus itself (Rosenfeld, M. A., et al., Science 252:431434 (1991)). Because the adenovirus functions in an extrachromosomal fashion, the recombinant adenovirus does not have the theoretical problem of insertional mutagenesis.

The instant invention also provides various methods for making and using the above-described genetically-modified cells. In particular, the invention provides a method for genetically modifying cell(s) of a mammalian recipient ex vivo and administering the genetically modified cells to the mammalian recipient. In one embodiment for ex vivo gene therapy, the cells are autologous cells, i.e., cells isolated from the mammalian recipient. As used herein, the term “isolated” means a cell or a plurality of cells that have been removed from their naturally-occurring in vivo location. Methods for removing cells from a patient, as well as methods for maintaining the isolated cells in culture are known to those of ordinary skill in the art.

The instant invention also provides methods for genetically modifying cells of a mammalian recipient in vivo. According to one embodiment, the method comprises introducing an expression vector for expressing a heterologous gene product into cells of the mammalian recipient in situ by, for example, injecting the vector into the recipient.

In one embodiment, the preparation of genetically modified cells contains an amount of cells sufficient to deliver a therapeutically effective dose of the therapeutic agent to the recipient in situ. The determination of a therapeutically effective dose of a specific therapeutic agent for a known condition is within the scope of one of ordinary skill in the art without the need for undue experimentation. Thus, in determining the effective dose, one of ordinary skill would consider the condition of the patient, the severity of the condition, as well as the results of clinical studies of the specific therapeutic agent being administered.

If the genetically modified cells are not already present in a pharmaceutically acceptable carrier they are placed in such a carrier prior to administration to the recipient. Such pharmaceutically acceptable carriers include, for example, isotonic saline and other buffers as appropriate to the patient and therapy.

The genetically modified cells are administered by, for example, intraperitoneal injecting or implanting the cells or a graft or capsule containing the cells in a target cell-compatible site of the recipient. As used herein, “target cell-compatible site” refers to a structure, cavity or fluid of the recipient into which the genetically modified cell(s), cell graft, or encapsulated cell expression system can be implanted, without triggering adverse physiological consequences. More than one recombinant gene can be introduced into each genetically modified cell on the same or different vectors, thereby allowing the expression of multiple therapeutic agents by a single cell.

The instant invention further embraces a cell graft. The graft comprises a plurality of the above-described genetically modified cells attached to a support that is suitable for implantation into a mammalian recipient. The support can be formed of a natural or synthetic material.

According to another aspect of the invention, an encapsulated cell expression system is provided. The encapsulated system includes a capsule suitable for implantation into a mammalian recipient and a plurality of the above-described genetically modified cells contained therein. The capsule can be formed of a synthetic or naturally-occurring material. The formulation of such capsules is known to one of ordinary skill in the art. In contrast to the cells that are directly implanted into the mammalian recipient (i.e., implanted in a manner such that the genetically modified cells are in direct physical contact with the cell-compatible site), the encapsulated cells remain isolated (i.e., not in direct physical contact with the site) following implantation. Thus, the encapsulated system is not limited to a capsule including genetically-modified non-immortalized cells, but may contain genetically modified immortalized cells.

The following examples are intended to illustrate but not limit the invention.

### EXAMPLES

### Example 1

### Adenovirus 30 Fiber Does Not Mediate Transduction via the Coxackie Adenovirus Receptor (CAR)

The following experiments were performed to test if Ad30 fiber bound CAR. For these experiments the Ad30 fiber was first cloned and sequenced. Ad30 fiber interactions were then tested with CAR, using both Ad30 wild-type virus and a recombinant adenovirus type 5 with its native fiber replaced by that of Ad30.

Cells and viruses. Human embryonic kidney (HEK 293) cells were maintained in Dulbecco modified Eagle medium (DMEM) with 10% fetal calf serum (FCS), 1% glutamine, and 1% penicillin-streptomycin. Chinese hamster ovary (CHO) cells from the American Type Culture Collection (ATCC) were maintained in DMEM F12 medium supplemented with 1% penicillin-streptomycin and 10% FCS. Ad30 (VR-273) was purchased from the ATCC and subsequently amplified by infection of HEK 293 cells. Viral particles were banded in CsCl gradients, dialyzed, and stored in 100 μl aliquots at −30° C. Ad5CMVhCAR (AdhCAR) and Ad5CMVntlacZ (Ad5lacZ) was produced by the Univerisity of Iowa Gene Transfer Vector Core.

Sequencing Ad30 fiber protein. Viral DNAs from purified Ad30 particles was isolated by standard protease treatment and ethanol precipitation methods. Degenerate primers to the 5′ and 3′ ends of the fiber gene were designed by means of comparison of the known sequences of four D-serotype viruses, adenovirus types 8, 9, 15 and 17. They are 5′-CGGGATCCGCCACCATGTCAAAGAGGCTCCGG-3′ (AdDfiberF; SEQ ID NO:21) and 5′-CGGGATCCTRATTCTTGGGCYATATAGG-3′ (DfiberR; SEQ ID NO:22). The fiber gene was completely sequenced in both directions.

Construction of Ad5GFPf30. The endogenous fiber sequence of Ad5 (nt 31042 to 32787) was replaced with Ad30 sequence by overlapping PCR. The Ad30 fiber was amplified such that it contained the first 147 base pairs of Ad5 tail (bp 31042 to 31189). Overlapping primers specific for the tail/shaft boundary containing 19 bps of Ad5 and 18 bps of Ad30 sequence were generated. In the first phase of the overlapping PCR, two DNA fragments corresponding to the Ad5 tail region and the Ad30 shaft and knob regions were amplified. The tail was generated using Ad5fiber for BamH1 (5′-CGCGGATCCGCGATGAAGCGCGCAAGA-3′; SEQ ID NO:23; bp 31042 to 31189) and 17Ad5overtail (5′-GATTGGGTCAGCCAGTTTCAAAGAGAGTACCCCAGG-3′; SEQ ID NO:24) with Biolase. The shaft/knob was amplified with the 5Ad17overtail (5′-CCTGGGGTACTCTCTTTGAAACTGGCTGACCCA-3′; SEQ ID NO:25) and Ad30fRevSpe1 (5′-AAAACTAGTTCATTCTTGGGCGATATA-3′ SEQ ID NO:26). Primers to the 5′ and 3′ ends were designed to incorporate the restriction enzyme recognition sites, BamHI and SpeI respectively. After 30 PCR cycles, the Ad5 tail and Ad30 shaft/knob products were purified by agarose gel electrophoresis, then mixed and the mixture was used as a template with Ad5fiber for BamH1 and Ad30fRevSpe1 primers to amplify the entire chimeric Ad5/30 fiber. The 1119-bp-long chimeric Ad5/30 fiber product, containing the Ad5 tail and the Ad30 shaft and knob domains, was purified by agarose gel electrophoresis, digested with Nde1 and Spe1 and ligated into a plasmid containing bases 29509-33096 of the Ad5 genome, pBS/B2HI. The resulting plasmid, pBS-5/30, linearized by Not1 and BamH1, and pTG3602/RSVeGFP/Swa1, linearized by Swa1 to drive homologous recombination in the region of fiber, were used to co-transform the RecA⁺ E. coli strain BJ5183. The resulting recombinants were screened by PCR and direct sequencing. Positive recombinants contained the entire Ad5 genome, flanked by PacI sites with the following modifications—replacement of the E1 region by the respiratory syncytial virus-enhanced green fluorescent protein (RSVeGFP) expression cassette and replacement of the endogenous Ad5 fiber with the chimeric Ad5/30 fiber. This plasmid was then digested by PacI and transfected into HEK 293 cells for the production of viral particles as described previously (Anderson, R. D. et al., 2000. Gene Ther. 7(12):1034-1038). Cytopathic effect (CPE) was evident 14 days post-transfection in 60 mm diameter dishes of HEK 293 cells. Lysates of Ad5RSVeGFPf30 (Ad5GFPf30) were used for further amplification. CPE was evident 40 h post-infection. Virus was harvested and purified by standard methods. The Ad5RSVeGFP control virus (Ad5GFP), with non-recombinant fiber, was similarly generated. Both viruses were analyzed by plaque assay on HEK 293 cells or A549 cells. Using this method the titer of Ad5GFPf30 was 4×10⁹ pfu/ml and Ad5GFP was 1.5×10¹⁰ pfu/ml. For all experiments, equivalent particle concentrations were used.

Analysis of recombinant fiber. Purified Ad5GFP and Ad5GFPf30 (2×10¹⁰ particles) were boiled at 95° C. for 15 min in Laemli buffer and fractionated by SDS-PAGE. Proteins were transferred to nitrocellulose membranes, blocked with 5% skim milk in PBS-0.1% Tween for 1 h at RT, and incubated with a monoclonal antibody to the N-terminus of Ad5 fiber (4D2.5, diluted 1 to 2500 in PBS-0.1% Tween overnight at 4° C. The membrane was then washed four times for 5 minutes with PBS-0.1% Tween and incubated with peroxidase-conjugated goat anti-mouse secondary (Jackson ImmunoResearch Laboratories, Inc., West Grove, Pa.) diluted 1 to 2500 in PBS-0.1% Tween for 1 h at room temperature. Membranes were washed as previously done and then developed with enhanced-chemiluminescence (ECL) reagent (Amersham Pharmacia, Piscataway, N.J.) according to the manufacturer's directions.

Labeling of Ads with methyl-3H-thymidine. Ad5GFP, Ad5GFPf30 and Ad30 wildtype were labeled with [methyl-3H] thymidine as described (Roelvink, P. W. et al 1996. J. Virol. 70(11):7614-7621). Briefly, 150-mm² dishes were seeded with 2.5×10⁷ HEK 293 cells in 15 ml of DMEM-10% FCS. Twenty-four hours later these cells were infected with recombinant adenovirus at an multiplicity of infection (MOI) of 50 or higher. Ten hours post-infection, 1.25 mCi of [methyl-³H] thymidine (Amersham, Arlington Heights, Ill. 3.03 TBq/mmol-82.0 Ci/mmol) was added to the medium, and cells were further incubated at 37° C. until ˜30 hours post-infection. Then cells were harvested, pelleted, washed once with phosphate-buffered saline (PBS), and resuspended in 1 ml of PBS. Virus was released from the cells by four freeze-thaw cycles. Cell debris was removed by centrifugation, and viral material was subjected to ultracentrifugation in a CsCl gradient and subsequent dialysis as previously against PBS, 3% sucrose. Virion-specific radioactivity, measured by liquid scintillation (Packard Tri-Carb 1500 Liquid Scintillation Analyzer), ranged from 9×10⁻⁶ to 4×10⁻⁵ cpm per virion.

CAR Infection Studies. CHO cells were plated 24 h prior to infection at a density of 3×10⁵ cells per 60 mm dish. CHO cells were transfected with Ad5lacZ or Ad5hCAR previously precipitated with CaPᵢ. Ad5lacZ or Ad5hCAR (4 μl of 10¹² particles/ml) was added to 1 ml of MEM, precipitated by the addition of 25 μl of 1 M CaCl₂, lightly vortexed, and then incubated at RT for 20 min. Medium was removed for CHO cells, and 1 ml of MEM containing the Ad-CaPᵢ precipitant was added to each dish. Cells were incubated with precipitated Ad5lacZ or Ad5hCAR at 13,000 particles/cell for 30 min. at 37° C., washed with fresh medium, and then incubated with 3 ml of fresh medium for an additional 24 h at 37° C. Twenty-four hours after CaPᵢ-mediated infections, CHO cells were incubated with 500 particles of Ad5GFP or AD5GFPf30/cell for 30 min. at 37° C. Cells were washed and incubated an additional 24 h at 37° C. before fluorescence-activated cell sorter (FACS) analysis.

FACS analysis. Infected cells were detached from dishes by incubation with trypsin for five minutes at 37° C. followed by pelleting, resuspension in media with propidium iodide (PPI), and FACS analysis for GFP expression. FACs were performed on a Becton Dickinson flow cytometer (San Jose, Calif.) equipped with a 488-nm argon laser. To detect CAR expression, cells were detached from dishes with EDTA, spun down and resuspended in 1% FBS/PBS at 2×10⁵ cells/ml and incubated with monoclonal antibodies (MAbs) against CAR for 45 min at 37° C. Cells were pelleted, washed and resuspended with R-phycoerythrin-conjugated goat anti-mouse secondary antibodies (Jackson ImmunoResearch 115-116-146) for 45 min. at 4° C., prior to FACS analysis.

Binding assays. The studies were performed as previously described (Wickham, T. J. et al. 1993. Cell 73:309-319). CHO cells were transfected with AdhCAR previously prepared with CaPᵢ as described above. Forty-eight hours after CAR transfection, transfected or non-transfected cells were incubated for 1 h with equal amounts of ³H-labeled Ad5GFP, Ad5GFPf30 or Ad30 wild-type particles on ice in 1 ml of ice-cold MEM. Cells were washed twice with 1 ml ice-cold MEM, harvested with trypsin and the cell-associated radioactivity was determined by scintillation counting.

Discussion

Ad30 fiber was cloned, sequenced and analyzed for its ability to bind CAR. Direct amino acid sequence comparison of Ad30 fiber with that from Ad5 demonstrated disparate as well as highly homologous regions. Sequences within the tail were most conserved, followed by the knob region. Within the knob, there was surprising conservation of amino acid sequences, and in the region of knob shown to be important in CAR binding by competitive or cell-free surface plasmon resonance assays (Kirby, I. et al. 2000. J. Virol. 74(6):2804-2813; Roelvink, P. W. et al. 1999. Science 286:1568-1571), there were homologous or conservative amino acid substitutions in seven of nine instances. It was presumed that this would allow Ad30 fiber-directed binding of adenoviruses to CAR. When tested using wild-type Ad30, or a recombinant Ad5 virus with its endogenous fiber shaft and knob sequences replaced with those from Ad30, it was found that Ad30 fiber does not bind CAR.

Within the knob, residues Ser₄₀₈, Pro₄₀₉, Lys₄₁₇, Lys₄₂₀, Tyr₄₇₇ and Arg₄₈₁, shown to be important in CAR-binding, were found to be identical between Ad5 and Ad30. Mutations at Ala₄₀₆, Arg₄₁₂, and Leu₄₈₅ of Ad5, diminish CAR binding. However, these substitutions (Asp₄₀₆, Lys₄₁₂, and Asp₄₈₅) are also present in Ad9 and/or Ad17, indicating that these changes alone are probably not sufficient for inhibiting binding to CAR.

More distinctive between Ad30 and the CAR-binding viruses are the amino acids surrounding these critical regions. The Ala₄₀₆ in Ad5, or the Asp₄₀₆ in Ad30, Ad9, and Ad17 is an example. In Ad5, 9, and 17, there is an invariable proline at amino acid position 405, and serine or threonine at position 407. Ad30 possesses a leucine and proline at 405 and 407, respectively. And although Tyr₄₇₇ is conserved in Ad30, Ad5, Ad9 and Ad17, the residues flanking Tyr₄₇₇ are distinct when comparing Ad30 to the CAR-binding fibers.

The Ad30 fiber shaft, similar to other D-serotype viruses, was found to be short relative to the Ad5 shaft. Nonetheless, significant homology was found for the first 70 amino acids, as well as regions near the hinge regions, notably from residues 378-400. Serotype B viruses Ad35 and Ad3 also have short shaft lengths relative to Ad5. Studies by Shayakhmetov and Leiber suggest that shaft length is important in CAR binding for Ad5 and Ad9; when Ad5 or Ad9 knobs were placed onto shorter shafts, CAR interactions were impaired (Shayakhmetov, D. M. and A. Lieber. 2000. J. Virol. 74(22):10274-10286). However, fiber length was not critical for adenoviruses entering cells via a non-CAR dependent pathway as was evident for Ad35. The rationale for this difference is suggested to be a charge repulsion between the negatively charged region within hypervariable region 1 of Ad5 hexon and acidic proteins on the cell surface.

Ad30 wildtype and the chimeric AdGFPf30 viruses were propagated in HEK 293 cells at similar levels to each other, but less efficiently relative to Ad5-fiber expressing viruses. It is possible that Ad30, like Ad9, is more dependent on penton base interactions with cell surface integrins for viral entry, although the presence of RGD motifs in the Ad30 penton base is currently unknown. Similarly, the shorter shaft of the Ad30 fiber may allow for more appropriate contact between the Ad5 penton base on Ad5GFPf30 capsids and cell surface αᵥ-integrins.

In summary, the fiber from Ad30, a D-serotype virus, was sequenced and analyzed and found to possess characteristics different from previously reported D-serotype virus fibers. It does not bind CAR although it retains many of the sequences within knob region predicted to be involved in CAR binding. The Ad30 fiber, therefore, is useful for directing gene transfer to non- or low-CAR expressing cells, either directly, or through specific targeting.

### Example 2

### Recombinant Human Adenovirus 5 Containing Transferrin Receptor Sequences

In this study the feasibility of targeting recombinant viral vectors to the hTfR to transduce the BME was tested. An available phage display library was used to identify motifs specific to the hTfR, and showed that these motifs did not inhibit fiber trimerization. In many cases fiber sequences modified to express the identified motifs in the HI loop allowed for the production of viable recombinant virus. Importantly, the sequences retained their ability to bind to hTfR and direct gene transfer to hTfR-expressing cells.

It was found that the addition of peptide sequences into the HI loop of fiber was not uniformly well tolerated, even among sequences that were highly similar with regard to charge and side group. For instance, viruses containing the peptide B2 motif were capable of being amplified to some degree, while those containing the similar B6 motif (Table 5) had greatly improved growth properties. Similarly, the B4 sequence was not amenable to virus production, while the B3-containing virus grew quite well. Because it was found that fiber trimerization was not affected, the block in virus production could result from steric hindrance of CAR binding. To overcome this problem for Ad5GFPB4HI, Ad5GFPB9HI, and Ad5GFPB10HI, amplification could be done using HEK 293 cells modified to express a surrogate receptor.

TABLE 5

Peptide motifs and their effects on recombinant
adenovirus production

| | | |
|---|---|---|
| | | Virus |
| Peptide | Epitope | production^{a} |

| | | | |
|---|---|---|---|
| | | Control | + + + + |
| IEAYAKKRK | (SEQ ID NO:10) | B1 | + + |
| GHKVKRPKG | (SEQ ID NO:9) | B2 | ± |
| KDKIKMDKK | (SEQ ID NO:11) | B3 | + + + + |
| G KNKIPKSPK LGS^{b} | (SEQ ID NO:27) | B4 | − |
| G GKGPKWMR LGS | (SEQ ID NO:28) | B5 | + + + |
| G GHKAKGPRK LGS | (SEQ ID NO:29) | B6 | + + + + |
| G VIAKIKKPK LGS | (SEQ ID NO:30) | B7 | + + + |
| G KWKTPKVRV LGS | (SEQ ID NO:31) | B8 | + |
| G LQAKKKRPK LGS | (SEQ ID NO:32) | B9 | − |
| G VEAKGHKKK LGS | (SEQ ID NO:33) | B10 | − |

Interestingly, earlier reports (Douglas et al., Nat. Biotechnol. 17:470-475 1999); Einfeld et al., J. Virol. 73:9130-9136 (1999)) demonstrated the feasibility of using an epitope cloned into the HI loop or carboxy terminus of fiber, or penton base, to allow adenovirus binding and entry mediated by a membrane-bound ScFv directed to that epitope. The present data suggest that other ligand-receptor pairs may not be sufficient to support viral production when CAR-fiber interactions are impaired. HEK 293 cells express high levels of hTfR based on Western blot analyses and immunofluorescence microscopy. However, production of B2 and B8 motif-modified adenoviruses was quite difficult even though binding to the hTfR was retained in the context of fiber and the intact virion.

The ability of a membrane-bound ScFv versus that of a recycling receptor-ligand pair (TfR-Tf) to serve as pseudoreceptor suggests that differences in recycling and internalization may be important. Data presented by Leopold and colleagues suggest that Ad5 escapes the endosome very early after internalization, probably before endosome-endosome fusion (Leopold et al., Hum. Gene Ther. 11:151-165 (2000)). It is possible that some HI loop-modified adenoviruses are impaired in their ability to direct early endosomal release, thereby allowing for some recycling and release of virus at the cell surface.

The B6 and B8 targeting motifs in recombinant adenovirus significantly improved gene transfer to hTfR⁺ CHO cells, T24 cells, and human BME cells. Also, both were more effective than the other motifs tested. The B2 epitope in Ad5GFPB2HI, which was only tested on hTfR⁺ CHO cell lines, has an amino acid sequence similar to that of B6. When cloned into adenovirus, both Ad5GFPB2HI and Ad5GFPB6HI resulted in an approximately 34-fold increase in gene transfer. The differences between the B2 and the B6 motifs are a valine in the +4 position (versus alanine), an arginine in the +6 position (versus glycine), a lysine in the +8 position (versus arginine), and a glycine in the +9 position (versus lysine). In cloning the B5-B8 epitopes into fiber, an amino-terminal glycine and a carboxyl-terminal LGS linker (relative to the nonapeptide sequence) was added. Thus, the linker did not appear to further improve or inhibit hTfR targeting. However, and importantly, the addition of the linker did facilitate virus growth as previously discussed.

At the outset of the studies, it was expected that epitopes isolated by ligand elution would yield better results when cloned into the HI loop of fiber compared to those identified using acid wash. The present data show that the B3, B5, B7, and B8 sequences, identified by acid elution, facilitated adenovirus-mediated transduction to hTfR-expressing cells ca. 1.4-fold less effectively (on average) than did B6, B11, and B2. However, both B1 and B2 were identified by acid and transferrin elution, suggesting that there is only modest correlation between elution parameters and the ability of that motif to facilitate receptor targeting, at least for the hTfR.

Targeting recombinant virus vectors to a receptor expressed at high density on BME cells is a first step toward testing if the vascular system can be used to facilitate a global distribution of enzyme to the CNS for inhibition or reversal of neurodegeneration. If sufficient levels of transduction to the endothelia could be accomplished, basolateral secretion would provide a source of enzyme to an extensive area of the brain. In brains of larger animal models or in humans, such an approach would bypass the requirement of multiple parenchymal injections, which could result only in small, nonoverlapping spheres of correction.

In summary, the present data suggest that several short motifs, when cloned into the HI loop of fiber, can support hTfR-targeted transduction with recombinant adenovirus vectors.

Cell culture. All media were supplemented with 10% fetal bovine serum (FBS) unless otherwise indicated. Human embryonic kidney cells (HEK 293) were maintained in Dulbecco's modified Eagle's medium (DMEM). Chinese hamster ovary cell (CHO) cells expressing human transferrin receptor (a kind gift from Martin Lawrence, Harvard University, Cambridge, Mass.), were maintained in F-12 Nutrient Mixture. HeLa cells (obtained from the American Type Culture Collection (ATCC), Rockville, Md.) were grown in minimal essential medium (MEM). The human prostate cancer cell line T24 was also from the ATCC and was maintained in 1640 medium. Human BME cells (kindly provided by Jay Nelson, Oregon Health Sciences University) were grown in 10% human AB serum (Sigma-Aldrich, St. Louis, Mo.) and in EBM (Clonetics, Walkersville, Md.).

Phage screening. Ten microliters of amplified nonapeptide phage library (a generous gift from Al Jesaitis, Montana State University) was screened against the purified extracellular domain of hTfR (kindly provided by Martin Lawrence, Harvard University) coated on 96-well microtiter plates in 100 μl of 0.05 M carbonate buffer (pH 9.6). In the first panning, hTfR was coated at 100 μg/ml. Subsequent pannings were done with decreasing concentrations of hTfR for increased stringency (10 and 1 μg/ml for the second and third pannings, respectively). Bound phage were eluted with low-acid buffer (0.1 M glycine, pH 2.2) or ligand (iron-loaded human transferrin; Sigma-Aldrich) in TBS buffer (50 mM Tris-Cl, pH 7.5; 150 mM NaCl). After three successive rounds of panning and amplification, clones were picked and sequenced as described elsewhere.

Phage and peptide binding assays. The extracellular domain of the hTfR was coated on 96-well microtiter well plates in 150 μl of 0.05 M carbonate buffer (pH 9.6) overnight (ON). The plates were then blocked (200 μl, 3% bovine serum albumin [BSA] for 2 h), washed, and incubated with purified phage (10¹⁰ phage) B1, B2, or a sequenced random clone from the library for 1 h at room temperature. Plates were washed and incubated with a rabbit anti-fd bacteriophage biotin conjugate (Sigma-Aldrich) directed against the M13 phage. Plates were developed using extravidin-peroxidase conjugates and diaminobenzidine (DAB) and then read at 490 nm using a microplate reader (Molecular Devices, Sunnyvale, Calif.). Data are presented as the mean of triplicates±the standard error of the mean (SEM). The experiments were repeated three times.

B2 peptide binding was tested for specificity for hTfR in two separate assays. For both, hTfR was first coated onto 96-well plates. In one assay, plates were coated ON with 3.75 μl of hTfR in 150 μl, blocked, and washed, followed by the addition of 100 μl of peptide B2 conjugated to biotin (Genosys Biotechnologies, Inc., Woodlands, Tex.) at a concentration range of 500 to 62.5 μg/ml. In the second assay plates were coated with 150 μl of hTfR (range, 25 to 1.6 μg/ml). After overnight coating, wells were blocked and then incubated with B2-labeled biotin (25 μg). Plates were developed and read as described above. Data are presented as the means of triplicates±the SEM. The experiments were repeated three times.

Construction of recombinant plasmids. To facilitate the generation of HI loop-modified viruses, the Ad5 fiber gene was first cloned into the vaccinia virus expression vector pTM1 (a kind gift from Michael J. Welsh, University of Iowa) by PCR amplification. This plasmid was designated pTM1Ad5fiber. Unique restriction sites and the B2 sequence were then introduced into fiber. To accomplish this, two pairs of primers, F1 (5′-AGAAATGGAGATCTTACTGAAGGC-3′; SEQ ID NO:37) and R1 (5′-CCCCTTCGGCCTCTTCACCTTATGACCAGTTGTGTCTCCTGTTTCCTGT GTACC-3′; SEQ ID NO:34) and also F2 (5′-GGTCATAAGGTGAAGAGGCCGAAGGGGCCAAGTGCATACTCTATGTC ATTTTCA-3′; SEQ ID NO:35) and R2 (5′-AACCCCGGGACTAGTCTATTCTTGGGCAATGTATGAAAAAGTGTA-3′; SEQ ID NO:36), were used to amplify a 210- and a 100-bp fragment of Ad5 fiber using purified virus genomic DNA as a template. The reaction products were gel purified and mixed, and contiguous sequences were generated by overlapping PCR using primers F1 and R2. The PCR amplification product contained a unique BglII site at the 5′ end and 3′ SpeI and SmaI sites. The restricted fragment was cloned into BglII- and SmaI-restricted pTM1Ad5 fiber. The resultant plasmid was named pTM1Ad5fiber/B2HI. All other motifs were similarly introduced using specific primer pairs. These plasmids were named pTM1Ad5fiber/B1HI, etc., and were used in in vitro expression systems to analyze the effects of the epitopes on fiber trimerization or binding to hTfR.

A shuttle was developed to allow insertion of modified HI loops into Ad5 fiber sequences. First, pTG1696 (obtained from Transgene S. A., Strasbourg, France) was cut by NotI and SpeI to remove approximately 8,000 bp of the first half of the plasmid. The plasmid was reclosed to generate pTGSN53 and contained adenovirus sequences from bp 29510 to 35935. pTM1Ad5fiber/B2HI was cut by SphI and SmaI, and the fragment was cloned into pTGSN53 to obtain pTGSS/B2HI plasmids. To facilitate homologous recombination in Escherichia coli, more than 1.0 kb of adenovirus sequence was introduced at the 3′ end of the fiber sequence. The primers Fbs (5′-CCCACTAGTATCGTTTGTGTT-3′; SEQ ID NO:38) and Rbs (5′-AAAGGATCCAGATCTGTTTGTCACGCCGCG-3′; SEQ ID NO:39) were used to amplify a fragment containing SpeI and BamHI restriction sites (underlined) at the 5′ and 3′ ends of the fragment, respectively, using Ad5 genomic DNA as a template. The PCR product was cut by BamHI and SpeI and then cloned into pTGSS/B2HI. The resulting plasmid was designated pBS/B2HI. pBS/B2HI contains the hTfR-targeting peptide B2 in the HI loop of Ad5 fiber and the novel SpeI site at the end of fiber coding sequence. Moreover, this plasmid also has greater than 1 kb of flanking Ad5 DNA sequence on either side of the fiber. pBS/B2HI will be useful for the cloning of any identified motif into the HI loop or for the generation of chimeric fiber sequences.

For plasmids pBS/B1HI, pBS/B3HI, etc., overlapping PCR was used to generated fragments containing motifs B1 and B3 to B10. These fragments were cut with SpeI and SphI and cloned into similarly cut pBS/B2HI to generate pBS/BxHI.

A full-length adenovirus backbone plasmid for recombination with pBS/BxHI was also generated. The plasmid pTG3602 (Transgene S.A.), which contains a wild-type fiber sequence, was modified to contain a unique SwaI site in fiber to facilitate homologous recombination. To accomplish this, pTG3602 was partially digested by NdeI and then ligated with an NdeI linker 5′-TACGCCCCATTTAAATGG-3′ (SEQ ID NO:40) containing an SwaI site (underlined). The plasmid was designated pTG3602/SwaI. pTG3602/SwaI was cut with ClaI and cotransformed into E. coli BJ5183 with ScaI-linearized pacAd5RSVGFP to generate pTG3602RSVGFP/SwaI. Positive clones were screened by enzyme digestion and sequencing. A 4.6-kb BamHI and NotI restriction fragment was liberated from pBS/BxHI and cotransformed with SwaI-linearized pTG3602RSVGFP/SwaI into E. coli BJ5183 to generate pTG3602RSVGFP/BxHI (pAd5GFPBxHI; see FIG. 4).

Viruses. Ad5GFP, with GFP under the control of Rous sarcoma virus (RSV) promoter, was from the Gene Transfer Vector Core, University of Iowa. hTfR-targeting viruses were generated by transfection of HEK 293 cells with PacI-digested peptide-modified virus vectors (Chartier et al, J. Virol. 70:4805-4810 (1996)). pAd5GFPBxHI (10 to 15 μg) were digested with 16 U of PacI at 37° C. for 2 h. The DNA was precipitated and transfected into HEK 293 cells using calcium phosphate. After 5 to 10 days, the lysates were harvested and further propagated on HEK 293 cells. Finally, viruses were purified by centrifugation in CsCl gradients according to standard protocols. Virus particle titers were determined spectrophotometrically. The viruses were named Ad5GFPBxHI, where “x” is the epitope number (B1, B2, etc.).

Fiber trimerization assays. Fifty to 70% confluent HeLa cells in 150-mm plates were rinsed with serum-free MEM and then incubated with vaccinia virus VTF7-3 at a multiplicity of infection of 10 (a kind gift from Michael J. Welsh, University of Iowa) in 3 ml of MEM at 37° C. for 1 h. Cells were washed and then transfected by pTM1Ad5fiber/BxHI plasmids or pTM1Ad5fiber (wild-type fiber) (10 μg) using Lipofectin (Gibco). After transfection, cells were rinsed and incubated in 30 ml of MEM-10% FBS at 37° C. The lysates were harvested 16 to 24 h later for trimerization assays. A 10-μl aliquot of lysate containing the recombinant proteins was subjected to reducing (31.25 mM Tris-Cl, pH 6.8; 1% sodium dodecyl sulfate [SDS]; 2.5% 2-mercaptoethanol [2-ME]; 10% glycerol) or nonreducing (the same except no 2-ME) conditions and fractionated by SDS-12% polyacrylamide gel electrophoresis (PAGE). The fractionated protein was transferred onto nitrocellulose membranes and probed by anti-fiber monoclonal antibody 4D2.5 (kindly provided by J. Engler, University of Alabama, Birmingham). The film was developed using an ECL Kit (Amersham Pharmacia Biotech, Piscataway, N.J.) according to the manufacturer's recommendations.

TfR binding assays with Bx targeting motifs in context of fiber. Protein G-conjugated agarose beads (60 μl; Pharmacia) were incubated with 15 μg of monoclonal antibody 128.1 directed against hTfR (generously provided by Ian Trowbridge, The Salk Institute, San Diego, Calif.). The 128.1-conjugated beads were resuspended in dilution buffer (10 mM Tris-Cl [pH 8.0], 140 mM NaCl, 0.1% Triton X-100, 0.1% BSA) and incubated with 15 μg of soluble hTfR for 1.5 h at 4° C. The complex was then incubated with 100 ill of vaccinia virus lysates containing wild-type or BxHI-modified fibers for 1.5 h at 4° C. The complexes were sequentially washed-twice with dilution buffer, twice with TSA buffer (10 mM Tris-Cl, pH 8.0; 140 mM NaCl), and once with 50 mM Tris-Cl (pH 6.8). The samples were denatured at 95° C. for 3 min, followed by microcentrifugation. The disrupted complex was fractionated by SDS-PAGE and transferred to nitrocellulose membranes. Membranes were probed using 4D2.5 monoclonal antibody directed against an epitope in the Ad5 fiber tail (Hong, J. Virol. 70:7071-7078 (1996)). The experiments were repeated three times.

Transduction of TfR⁺ cells. Experiments were carried out similarly to those described by Wickham et al (Nat. Biotech. 14:1570-1573 (1996)). hTfR⁺ CHO cells, human prostate cancer T24 cells, or human brain endothelial cells (10⁵) were incubated with Ad5GFPBxHI or Ad5GFP (4×10⁸ particles in 1 ml of DMEM-2% FBS) for 1 hour at 37° C. The cells were then washed three times with 2% FBS-DMEM, followed by incubation for 48 h at 37° C. The cells were then detached by trypsin and analyzed by using a fluorescence-activated cell sorter (FACS). In blocking experiments, hTfR+ CHO cells or T24 cells were incubated with human iron-loaded transferrin (40 μg/ml in 1 ml of DMEM-2% FBS) or soluble hTfR (15 μg/ml in 1 ml) for 30 min at 4° C. before the addition of virus. Human brain endothelial cells were incubated with human iron-loaded transferrin (40 μg/ml in 1 ml) for 30 min at 4° C. before the addition of virus. Data are presented as the means of triplicates±the SEM. The experiments were repeated three times.

Immunohistochemistry. T24 and BME cells were grown on 60-mm dishes, fixed in 2% paraformaldehyde, washed with PBS, and incubated with primary antibody (128.1 diluted 1:200 in PBS, 3% BSA, 0.3% Triton X-100, and 0.02% sodium azide) ON at 4° C. Plates were washed and incubated in rhodamine-conjugated goat anti-mouse (1:400) for 1 h at room temperature. Positive cells were visualized using an Olympus IX70 microscope, and images were captured using a SPOT RT digital camera.

### Example 3

### Recombinant Human Adenovirus 30 Containing Transferrin Receptor Sequences

Similar to the experiments described above for Ad5, a transferrin sequence is inserted into the HI loop of the Ad30 fiber protein or the HI loop of the Ad17 fiber protein.

### Example 4

### Recombinant Human Adenovirus Containing Different Receptor Sequences

The experiments described above in Examples 2-3 could be performed where other receptor-targeting sequences besides transferrin inserted into the HI loop of Ad5, Ad17 or Ad30 fiber protein. For example, receptor-targeting sequences that may be inserted into the HI loop of the fiber protein include, but are not limited to, tumor necrosis factors (or TNF's) such as, for example, TNF-alpha and TNF-beta; ApoB, which binds to the LDL receptor of liver cells; alpha-2-macroglobulin, which binds to the LRP receptor of liver cells; alpha-1 acid glycoprotein, which binds to the asialoglycoprotein receptor of liver; mannose-containing peptides, which bind to the mannose receptor of macrophages; sialyl-Lewis-X antigen-containing peptides, which bind to the ELAM-1 receptor of activated endothelial cells; CD34 ligand, which binds to the CD34 receptor of hematopoietic progenitor cells; CD40 ligand, which binds to the CD40 receptor of B-lymphocytes; ICAM-1, which binds to the LFA-1 (CD11b/CD18) receptor of lymphocytes, or to the Mac-1 (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; LFA-1, which binds to the ICAM-1 receptor of activated endothelial cells; NGF, which binds to the NGF receptor of neural cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; and the Fv antigen-binding domain of an immunoglobulin. Finally, one could modify the HI loop of fiber with the protein transductio domain of HIVTat to allow for fiber receptor independent entry and transduction of cells in vitro and in vivo.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. An adenoviral vector comprising an adenoviral backbone encoding an adenoviral fiber that does not bind coxsacki-adenovirus receptor (CAR) and an adenoviral fiber protein HI-loop operably linked to a receptor-targeting ligand to form a ligand/HI-loop chimeric protein, wherein the chimeric protein binds to a corresponding targeted receptor but does not bind CAR.

2. The vector of claim 1, wherein the backbone is an adenovirus 3, 5, 17, 30 or 35.

3. The vector of claim 1, wherein the HI-loop is from adenovirus 3, 17, 30 or 35.

4. The vector of claim 1, wherein the backbone and the HI-loop are from different types of adenovirus.

5. The vector of claim 4, wherein the backbone is from adenovirus 5 and the HI-loop is from adenovirus 30.

6. The vector of claim 1, wherein the ligand sequence is inserted into the HI-loop sequence.

7. The vector of claim 6, wherein the ligand is a tumor necrosis factor, transferrin, transferrin-receptor targeting ligand, ApoB, alpha-i acid, mannose-containing peptides, sialyl-Lewis-X antigen-containing peptides, CD34, CD40, ICAM-1, M-CSF, circumsporozoite protein, VLA-4, LFA-1, NGF, HIV gp120 or Class II MHC antigen, the LDL receptor binding region of the apolipoprotein E (ApoE) molecule, colony stimulating factor, insulin-like growth factors, Interleukins 1 through 14, glucose transporter targeting ligand, or the Fv antigen-binding domain of an immunoglobulin.

8. The vector of claim 6, wherein the sequence encoding the ligand is under the control of a suitable promoter.

9. The vector of claim 8, wherein the promoter is an adenoviral promoter or heterologous promoter.

10. The vector of claim 9, wherein the heterologous promoter is a cytomegalovirus (CMV) promoter, a respiratory syncytial virus (RSV) promoter, an inducible promoter, an MMT promoter, a metallothionein promoter, a heat shock promoter, an albumin promoter, endothelial-specific promoter or an ApoAI promoter.

11. The vector of claim 1, further comprising a polynucleotide sequence encoding a therapeutic agent operably linked to the chimeric sequence.

12. A chimeric protein comprising an amino acid sequence encoding an adenovirus fiber protein HI-loop operably linked to an amino acid sequence encoding a receptor-targeting ligand, wherein the chimeric protein binds to a corresponding targeted receptor but does not bind CAR.

13. The protein of claim 12, wherein the HI-loop is from adenovirus 3, 17, 30 or 35.

14. The protein of claim 12, wherein the ligand sequence is inserted into the HI-loop sequence.

15. The protein of claim 12, wherein the ligand is a tumor necrosis factor, transferrin, transferrin-receptor targeting ligand, ApoB, alpha-1 acid, a mannose-containing peptide, a sialyl-Lewis-X antigen-containing peptide, CD34, CD40, ICAM-1, M-CSF, a circumsporozoite protein, VLA-4, LFA-1, NGF, HIV gp120, Class II MHC antigen, LDL receptor binding region of apolipoprotein E (ApoE), colony stimulating factor, an insulin-like growth factor, Interleukins 1 through 14, glucose transporter targeting ligand, or an Fv antigen-binding domain of an immunoglobulin.

16. The protein of claim 12, operably linked to an amino acid sequence for a therapeutic agent.

17. An adenovirus particle comprising the vector of claim 1.

18. A mammalian cell containing the vector of claim 1.

19. The cell of claim 18, wherein the cell is human.

20. The cell of claim 18, wherein the cell is a prostate, brain or other neural, breast, lung, spleen, kidney, heart, liver, bone marrow, endothelial, activated endothelial, epithelial, keratinocyte, stem, hepatocyte, fibroblast, mesenchymal, mesothelial, parenchymal, vascular smooth muscle, gut enterocyte, gut stem, or myoblast cell.

21. The cell of claim 18, wherein the cell is a primary nucleated blood cell.

22. The cell of claim 21, wherein the blood cell is a leukocyte, granulocyte, monocyte, macrophage, T-lymphocyte, B-lymphocyte, totipotent stem cell, or tumor infiltrating lymphocyte (TIL cell).

23. The cell of claim 18, wherein the cell is a neural progenitor or stem cell.

24. A method of transducing cells lacking CAR comprising contacting the cells with the vector of claim 1.

25. The method of claim 24, wherein the cell is a neuronal, glial or epithelial cell.

26. The method of claim 24, wherein the cell is a human umbilical vein epithelial cell (HUVEC).

27. The method of claim 24, wherein the cell is a tumor cell.

28. The method of claim 27, wherein the tumor cell is from prostate, brain, breast, lung, spleen, kidney, heart, or liver.

29. The method of claim 25, wherein the cell is a neuroprogenitor or stem cell.

30. A method of transducing cells lacking CAR comprising contacting the cells with the adenovirus particle of claim 17.

31. A method of treating a genetic disease or cancer in a mammal comprising administering the vector of claim 1, the chimeric protein of claim 12, the adenovirus particle comprising the vector of claim 17, or the mammalian cell of claim 18.


SEQUENCE LISTING (40 sequences)

SEQ ID NO: 1 - protein, 371 aa, Adenovirus
MSKRLRVEDDFNPVYPYGYARNQNIPFLTPPFVSSDGFKNFPPGVLSLKLADPIAITNGDVSLKVGGGLTVEQDSGNLSVNPKAPLQVGTDKKLELALAPPFDVRDNKLAILVGDGLKVIDRSISDLPGLLNYLVVLTGKGIGNEELKNDDGSNKGVGLCVRIGEGGGLTFDDKGYLVAWNNKHDIRTIWTTLDPSPNCKIDIEKDSKLTLVLTKCGSQILANVSLIIVNGKFKILNNKTDPSLPKSFNIKLLFDQNGVLLENSNIEKQYLNFRSGDSILPEPYKNAIGFMPNLLAYAKATTDQSKIYARNTIYGNIYLDNQPYNPVVIKITFNNEADSAYSITFNYSWTKDYDNIPFDSTSFTFSYIAQE

SEQ ID NO: 2 - protein, 581 aa, Adenovirus
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTIWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 3 - protein, 362 aa, Adenovirus
MSKRLRVEDDFNPVYPYGYARNQNIPFLTPPFVSSDGFQNFPPGVLSLKLADPIAIVNGNVSLKVGGGLTLQDGTGKLTVNADPPLQLTNNKLGIALDAPFDVIDNKLTLLAGHGLSIITKETSTLPGLRNTLVVLTGKGIGTESTDNGGTVCVRVGEGGGLSFNNDGDLVAFNKKEDKRTIWTTPDTSPNCKIDQDKDSKLTLVLTKCGSQILANVSLIVVDGKYKIINNNTQPALKGFTIKLLFDENGVLMESSNLGKSYWNFRNENSIMSTAYEKAIGFMPNLVAYPKPTAGSKKYARDIVYGNIYLGGKPDQPVTIKTTFNQETGCEYSITFDFSWAKTYVNVEFETTSFTFSYIAQE

SEQ ID NO: 4 - protein, 366 aa, Adenovirus
MSKRLRVEDDFNPVYPYGYARNQNIPFLTPPFVSSDGFKNFPPGVLSLKLADPITIANGDVSLKVGGGLTLQEGSLTVDPKAPLQLANNKKLELVYVDPFEVSANKLSLKVGHGLKILDDKSAGGLKDLIGKLVVLTGKGIGTENLQNTDGSSRGIGISVRAREGLTFDNDGYLVAWNPKYDTRTIWTTPDTSPNCRIDKEKDSKLTLVLTKCGSQILANVSLIVVSGKYQYIDHATNPTLKSFKIKLLFDNKGVLLPSSNLDSTYWNFRSDNLTVSEAYKNAVEFMPNLVAYPKPTTGSKKYARDIVYGNIYLGGLAYQPVVIKVTFNEEADSAYSITFEFVWNKEYARVEFETTSFTFSYIAQQ

SEQ ID NO: 5 - protein, 322 aa, Adenovirus
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKCAKLGNGLKFNNGDICIKDSINTIWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 6 - protein, 319 aa, Adenovirus
MAKRARLSTSFNPVYPYEDESSSQHPFINPGFISPDGFTQSPNGVLSLKCVNPLTTASGSLQLKVGSGLTVDTTDGSLEENIKVNTPLTKSNHSINLPIGNGLQIEQNKLCSKLGNGLTFDSSNSIALKNNTIWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 7 - protein, 6 aa, Artificial Sequence
  A peptide motif.
AKXXXX

SEQ ID NO: 8 - protein, 6 aa, Artificial Sequence
  A peptide motif.
KXKXPX

SEQ ID NO: 9 - protein, 9 aa, Adenovirus
GHKVKRPKG

SEQ ID NO: 10 - protein, 9 aa, Adenovirus
IEAYAKKRK

SEQ ID NO: 11 - protein, 9 aa, Adenovirus
KDKIKMDKK

SEQ ID NO: 12 - protein, 9 aa, Adenovirus
KNKIPKSPK

SEQ ID NO: 13 - protein, 9 aa, Adenovirus
LQAKKKRPK

SEQ ID NO: 14 - protein, 9 aa, Adenovirus
VIAKIKKPK

SEQ ID NO: 15 - protein, 9 aa, Adenovirus
AIAKKHKWN

SEQ ID NO: 16 - protein, 9 aa, Adenovirus
VEAKGHKKK

SEQ ID NO: 17 - protein, 9 aa, Adenovirus
GHKAKGPRK

SEQ ID NO: 18 - protein, 9 aa, Adenovirus
KWKTPKVRV

SEQ ID NO: 19 - protein, 8 aa, Adenovirus
GKGPKWMR

SEQ ID NO: 20 - protein, 9 aa, Adenovirus
KWKLHGHIK

SEQ ID NO: 21 - DNA, 32 bp, Adenovirus
cgggatccgccaccatgtcaaagaggctccgg

SEQ ID NO: 22 - DNA, 28 bp, Adenovirus
cgggatcctrattcttgggcyatatagg

SEQ ID NO: 23 - DNA, 27 bp, Artificial Sequence
  A primer.
cgcggatccgcgatgaagcgcgcaaga

SEQ ID NO: 24 - DNA, 36 bp, Artificial Sequence
  A primer.
gattgggtcagccagtttcaaagagagtaccccagg

SEQ ID NO: 25 - DNA, 33 bp, Artificial Sequence
  A primer.
cctggggtactctctttgaaactggctgaccca

SEQ ID NO: 26 - DNA, 27 bp, Artificial Sequence
  A primer.
aaaactagttcattcttgggcgatata

SEQ ID NO: 27 - protein, 13 aa, Artificial Sequence
  A protein motif.
GKNKIPKSPKLGS

SEQ ID NO: 28 - protein, 12 aa, Artificial Sequence
  A protein motif.
GGKGPKWMRLGS

SEQ ID NO: 29 - protein, 13 aa, Artificial Sequence
  A protein motif.
GGHKAKGPRKLGS

SEQ ID NO: 30 - protein, 13 aa, Artificial Sequence
  A protein motif.
GVIAKIKKPKLGS

SEQ ID NO: 31 - protein, 13 aa, Artificial Sequence
  A protein motif.
GKWKTPKVRVLGS

SEQ ID NO: 32 - protein, 13 aa, Artificial Sequence
  A protein motif.
GLQAKKKRPKLGS

SEQ ID NO: 33 - protein, 13 aa, Artificial Sequence
  A protein motif.
GVEAKGHKKKLGS

SEQ ID NO: 34 - DNA, 54 bp, Artificial Sequence
  A primer.
ccccttcggcctcttcaccttatgaccagttgtgtctcctgtttcctgtgtacc

SEQ ID NO: 35 - DNA, 54 bp, Artificial Sequence
  A primer.
ggtcataaggtgaagaggccgaaggggccaagtgcatactctatgtcattttca

SEQ ID NO: 36 - DNA, 45 bp, Artificial Sequence
  A primer.
aaccccgggactagtctattcttgggcaatgtatgaaaaagtgta

SEQ ID NO: 37 - DNA, 24 bp, Artificial Sequence
  A primer.
agaaatggagatcttactgaaggc

SEQ ID NO: 38 - DNA, 21 bp, Artificial Sequence
  A primer.
cccactagtatcgtttgtgtt

SEQ ID NO: 39 - DNA, 30 bp, Artificial Sequence
  A primer.
aaaggatccagatctgtttgtcacgccgcg

SEQ ID NO: 40 - DNA, 18 bp, Artificial Sequence
  A primer.
tacgccccatttaaatgg